(19) United States
(12) Patent Application Publication
(10) Pub. No.: US 2021/0186547 A1
(43) Pub. Date: Jun. 24, 2021

(54) DEVICES, SYSTEMS, AND METHODS FOR LOCALLY ENGAGING TISSUE USING SUCTION

(71) Applicants: Ghassan S. KASSAB, San Diego, CA (US); Brad KLOS, San Diego, CA (US); George A. MANSFIELD, San Diego, CA (US); Matthew J. PHILLIPS, San Diego, CA (US); Adam ARIELY, San Diego, CA (US); Jeff LOOS, San Diego, CA (US); David G. MATSUURA, San Diego, CA (US)

(72) Inventors: Ghassan S. KASSAB, San Diego, CA (US); Brad KLOS, San Diego, CA (US); George A. MANSFIELD, San Diego, CA (US); Matthew J. PHILLIPS, San Diego, CA (US); Adam ARIELY, San Diego, CA (US); Jeff LOOS, San Diego, CA (US); David G. MATSUURA, San Diego, CA (US)

(21) Appl. No.: 17/054,368

(22) PCT Filed: May 15, 2019
(86) PCT No.: PCT/US2019/032524

§ 371 Date: Nov. 10, 2020

Related U.S. Application Data
(60) Provisional application No. 62/671,056, filed on May 14, 2018.

(51) Int. Cl. A61B 17/30; A61B 17/00

(52) U.S. Cl. CPC A61B 17/30; A61B 17/00234; A61B 2017/308; A61B 2017/00561; A61B 2017/00862; A61B 2017/00292; A61B 2017/00557; A61B 2017/00867

(57) ABSTRACT

Systems and methods for locally engaging tissue using suction. In at least one catheter device embodiment, the catheter device comprises an engagement catheter having a proximal end, a distal end, and a vacuum lumen therethrough operably coupling the proximal and distal ends; an elongated tube of a shape memory material having a plurality of elongated strips removed from a length of the elongated tube to form elongated shape memory tines at a distal end of the tube, wherein the shape memory tines flare outward away from a central axis to form a fluted distal end; and an elastomeric coating disposed over the elongated shape memory material tines to form a suction cup shape.

### PRIORITY

The present PCT application is related to, and claims the priority benefit of, U.S. Provisional Patent Application Ser. No. 62/671,056, filed May 14, 2018, the contents of which are incorporated herein directly and by reference in their entirety.

### BACKGROUND

Engagement catheters for use with suction to engage tissue within the mammalian body have become increasingly more important in the medical arts, as said catheters facilitate various medical procedures previously incapable of being performed without their use.

Embodiments of engagement catheters, providing improved functionality, stability, and the like, would be well received in the marketplace.

### BRIEF SUMMARY

An exemplary catheter device for engaging tissue using suction of the present disclosure comprises an engagement catheter having a proximal end, a distal end, and a vacuum lumen therethrough operably coupling the proximal and distal ends; a cone shaped suction cup disposed at the distal end of the engagement catheter to engage a tissue of interest, the cone shaped suction cup comprising a first layer of a material and a second layer of a material bonded together, wherein the first layer of material forms an inside wall of the cone shaped suction cup and wherein the second layer of material forms an outside wall of the cone shaped suction cup; and a plurality of rigid structural vanes, disposed between the first and second layers of material to stiffen the cone shaped suction cup; and a distal end vacuum port disposed inside of the cone shaped suction cup and operably coupled to the vacuum lumen to suction a tissue of interest.

In at least one embodiment, the plurality of rigid structural vanes are each inflatable. In at least one embodiment, the plurality of inflatable rigid structural vanes are inflated to deploy the suction cup. In at least one embodiment, the plurality of inflatable rigid structural vanes are disposed longitudinally spaced apart along a length of the suction cup. In at least one embodiment, the plurality of inflatable rigid structural vanes are disposed longitudinally and radially along a length of the suction cup. In at least one embodiment, the plurality of inflatable rigid structural vanes are disposed in a spiral around the circumference of the suction cup. In at least one embodiment, the device further comprises an inflatable rigid structural vane circumferentially disposed around a distal most end of the suction cup. In at least one embodiment, the device further comprises at least two large welded structural vanes disposed longitudinally along a length of the suction cup.

In at least one embodiment, the plurality of inflatable rigid structural vanes are disposed in a symmetrical looping pattern. In at least one embodiment, the device further comprises an inflatable annular area or balloon disposed on a distal most end of the suction cup. In at least one embodiment, the inflatable annular area comprises a tangent donut to engage a tissue of interest. In at least one embodiment, the plurality of inflatable rigid structural vanes increases surface area contact with tissue to improve suction strength and sealing. In at least one embodiment, the cone shaped suction cup is shaped by way of an elongated tube of a shape memory material having a plurality of elongated strips removed from a length of the elongated tube to form elongated shape memory tines at the distal end of the tube, wherein the shape memory tines flare outward away from a central axis to form a fluted distal end; wherein the first layer of the material and the second layer of the material are effectively one unitary layer of an elastomeric coating; and wherein the plurality of rigid structural vanes are configured as the elongated shape memory tines. In at least one embodiment, the suction cup comprises a flared distal end. In at least one embodiment, the suction cup has a uniform wall thickness or a tapered wall thickness. In at least one embodiment, the suction cup has a tapered wall thickness. In at least one embodiment, the suction cup has a wall thickness greater near the proximal end and thinner near the distal end. In at least one embodiment, the first and second layers of material are formed of a shape memory material. In at least one embodiment, the first and second layers of material are formed of an elastomeric material and the rigid structural vanes are formed of a shape memory material. In at least one embodiment, the plurality of rigid structural vanes have a uniform width and are disposed longitudinally spaced apart along a length of the suction cup. In at least one embodiment, the plurality of rigid structural vanes are disposed longitudinally and radially along a length of the suction cup. In at least one embodiment, the plurality of rigid structural vanes are disposed in a spiral around the circumference of the suction cup. In at least one embodiment, the device further comprises an rigid structural vane circumferentially disposed around a distal most end of the suction cup. In at least one embodiment, the plurality of rigid structural vanes are disposed in a symmetrical looping pattern around a circumference of the suction cup. In at least one embodiment, the plurality of rigid structural vanes are disposed in a zig-zag pattern around a circumference of the suction cup. In at least one embodiment, the plurality of rigid structural vanes are disposed in an elongated zig-zag pattern along a length of the suction cup.

In at least one embodiment, the plurality of rigid structural vanes extend longitudinally along a length of the suction cup and are connected together radially by thinner zig-zag linkages. In at least one embodiment, the suction cup comprises a pleated self-expanding skirt-shaped cone. In at least one embodiment, the first and second layers of material are elastomeric molded into pleats. In at least one embodiment, the plurality of rigid structural vanes further comprise radiopaque markers thereon. In at least one embodiment, each of the plurality of rigid structural vanes further comprises a radiopaque marker at its distal end. In at least one embodiment, the device further comprises a handle operably coupled to the proximal end of the engagement catheter and having a vacuum port operably coupled to the vacuum lumen for providing suction therethrough via an external vacuum source.

In at least one embodiment of a catheter system for engaging tissue using suction, the catheter system comprises an engagement catheter having a proximal end, a distal end, and a vacuum lumen therethrough operably coupling the proximal and distal ends; a handle operably coupled to the proximal end of the engagement catheter and having a vacuum port operably coupled to the vacuum lumen for providing suction therethrough via an external vacuum source; a cone shaped suction cup disposed at the distal end of the engagement catheter to engage a tissue of interest, the cone shaped suction cup comprising a first layer of a material and a second layer of material bonded together, wherein the first layer of material forms an inside wall of the cone shaped suction cup and wherein the second layer of material forms an outside wall of the cone shaped suction cup; and a plurality of rigid structural vanes, disposed between the first and second layers of material to stiffen the cone shaped suction cup; and a distal end vacuum port disposed inside of the cone shaped suction cup and operably coupled to the vacuum lumen to suction a tissue of interest; and a sleeve slidingly disposed around the engagement catheter and operably coupled to the handle, wherein movement of the sleeve relative to the engagement catheter will collapse or expand the suction cup.

In at least one embodiment, the suction cup is operable via the handle to retract into the sleeve. In at least one embodiment, the suction cup further comprises longitudinal pleats and to facilitate folding and retraction into the sleeve. In at least one embodiment, the suction cup further comprising a longitudinally pleated sleeve attached to an outer sheath at the proximal end and attached to an inner sheath at the distal end, wherein extension of the outer sheath, relative to a stationary inner sheath, will invert the longitudinally pleated sleeve to form the suction cup. In at least one embodiment, the sleeve further comprises a plurality of longitudinally disposed shape memory material linkages coupled to an outer sheath at the proximal end and coupled to an inner sheath at a distal end, wherein extension of the outer sheath, relative to a stationary inner sheath, will invert the longitudinally disposed shape memory material linkages to form the suction cup. In at least one embodiment, the engagement catheter further comprises a needle having a lumen sized and shaped to receive a guidewire therethrough.

In at least one embodiment, the system further comprises a needle for perforation of a fossa ovalis. In at least one embodiment, the handle further comprises detents and a locking pin operably configured to aid in retraction of the engagement catheter. In at least one embodiment, the handle further comprises a detent collet operably configured to extend the engagement catheter. In at least one embodiment, the vacuum port on the handle further comprises three-way stop cock to aid in turning suction on and off. In at least one embodiment, the handle further comprises a canted coil spring detent for expanding and collapsing the suction cup. In at least one embodiment, the handle further comprises dual hemostasis seals sized to provide a tight sealing connection while receiving guidewires, dilators or catheters. In at least one embodiment, the handle further comprises a thumb wheel for precise control over expansion and collapse of the suction cup. In at least one embodiment, the suction cup is operable to engage tissue when only partially expanded. In at least one embodiment, the handle is detachably coupled to the sleeve via a removable handle locking pin.

In at least one embodiment of a method of forming a catheter device for engaging tissue using suction, the method comprises selecting an elongated tubular section of shape memory material; laser cutting the elongated tubular section of shape memory material, to form: i) a plurality of elongated structural vanes separated by longitudinal slits laser cut into a distal most end of the tubular section; and ii) a plurality of tear-dropped shaped cut-outs within a distal most end of each elongated structural vane; heat setting the elongated tubular section of shape memory material to flare the plurality of elongated structural vanes away from a central longitudinal axis to form a fluted distal end; and sealing the fluted distal end with an elastomeric coating to form a cone shaped suction cup for engaging and suctioning tissue.

In at least one embodiment, heat setting the elongated tubular section comprises heat setting at 500° C.-550° C. In at least one embodiment, the plurality of elongated structural vanes form a framework upon which an elastomeric coating may be formed, the elastomer coating selected from the group consisting of: silicon or polyurethane. In at least one embodiment, the elongated tubular section of shape memory material further comprises elongated oval-shaped attachment slots around a circumference of the tubular section, at a most proximal end, for coupling to the catheter. In at least one embodiment, sealing the fluted distal end with an elastomeric coating, further comprising applying the elastomeric coating with a tapered wall thickness. In at least one embodiment, the fluted distal end further comprises two curved sections and a straight section.

In at least one embodiment, of a catheter device for engaging tissue using suction, the catheter device comprises an engagement catheter having a proximal end, a distal end, and a vacuum lumen therethrough operably coupling the proximal and distal ends; an elongated tube of a shape memory material having a plurality of elongated strips removed from a length of the elongated tube to form elongated shape memory tines at a distal end of the tube, wherein the shape memory tines flare outward away from a central axis to form a fluted distal end; and an elastomeric coating disposed over the elongated shape memory material tines to form a suction cup shape. In at least one embodiment, the shape memory material is Nitinol. In at least one embodiment, the elastomeric coating is polyurethane or silicone. In at least one embodiment, the device further comprises teardrop shaped elongated slots disposed in a distal most end of each of the shape memory material tines. In at least one embodiment, the device further comprises elongated oval shaped attachment slots disposed in the proximal end of the suction cup for attachment to the engagement catheter.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows a perspective view of an engagement catheter having a cone-shaped suction cup at its distal end, whereby the cone-shaped suction cup has one or more inflatable structural longitudinal vanes, according to an exemplary embodiment of the present disclosure;

FIG. 2 shows an exemplary structural geometric rendering of the cone-shaped suction cup of the engagement catheter of FIG. 1 having one or more inflatable structural longitudinal vanes, according to an exemplary embodiment of the present disclosure;

FIG. 3 shows a perspective view of an exemplary engagement catheter having a cone-shaped suction cup at its distal end with one or more inflatable structural longitudinal and radial vanes, according to an exemplary embodiment of the present disclosure;

FIG. 4 shows a cone-shaped suction cup 1830 having a greater number i.e., density, of longitudinally disposed structural vanes 6010;

FIG. 5 illustrates the cone-shaped suction cup 1830 having a spiral structural vane or vanes 6010 disposed radially around its circumference;

FIG. 6 illustrates a cone-shaped suction cup 1830 having longitudinally disposed structural vanes 6010 which are partially radially attached to one another;

FIG. 7 illustrates a cone-shaped suction cup 1830 having large longitudinally disposed structural vanes 6010 welded therein;

FIG. 8 illustrates a flute-shaped suction cup 1830 having a larger inflatable annular area, or balloon 6700, disposed on the distal end 6100 of the flute-shaped suction cup 1830;

FIG. 9 illustrates a cross sectional view of the embodiment of FIG. 8 having a larger inflatable annular area or balloon 6700 disposed on the distal end 6100 of the flute-shaped suction cup 1830;

FIG. 10 illustrates an engagement catheter 1810 having an even larger inflatable area or tangent donut 6900 positioned at distal end 720;

FIG. 11 illustrates a cross-sectional view of the inflated tangent donut 6900 of the embodiment of FIG. 10;

FIG. 12 shows a cross-sectional view of the deflated tangent donut 6900 of the embodiment shown in FIG. 10;

FIG. 13 illustrates a cross-sectional view of a different embodiment of an inflated tangent donut 6900 disposed on a distal end 720 of the engagement catheter 1810;

FIG. 14 illustrates a cross-sectional view of an embodiment of a deflated tangent donut 6900 of the embodiment shown in FIG. 13;

FIG. 15 illustrates an embodiment of an engagement catheter 1810 having a dip-molded cone-shaped suction cup 1830 disposed on its distal end 720;

FIG. 16 illustrates a cross-sectional view of the dip-molded cone-shaped suction cup 1830 having a tapered wall thickness;

FIG. 17 illustrates an embodiment of an engagement 1810 catheter having a collapsed cone-shaped suction cup 1830;

FIG. 18 illustrates an embodiment of the engagement catheter 1810 of FIG. 17 having a cone-shaped suction cup 1830 in an expanded or deployed configuration;

FIGS. 19 & 20 illustrate an exemplary embodiment of an engagement catheter 1810 having a Nitinol suction cup 1830 disposed on its distal end 720 in an expanded configuration;

FIGS. 21, 22, & 23 illustrate an exemplary embodiment of an engagement catheter 1810 having a Nitinol cone-shaped suction cup 1830 disposed on its distal end 720;

FIGS. 24 & 25 illustrate an exemplary embodiment of an engagement catheter 1810 having a Nitinol cone-shaped suction cup 1830 disposed on its distal end 720;

FIG. 26 illustrates an exemplary embodiment of an engagement catheter 1810 having a braided Nitinol flute 1830 on its distal end 720;

FIGS. 27, 28, & 29 illustrate an exemplary embodiment of an engagement catheter 1810 where the suction cup 1830 comprises a self-expanding pleated skirt shaped cone 1830;

FIG. 30 illustrates an exemplary embodiment of an engagement catheter 1810 having a pleated skirt shaped suction cup 1830 which can invert to then form the skirt shaped suction cup 1830;

FIG. 31 illustrates several exemplary embodiments of an engagement catheter 1810 having a pleated skirt shaped suction cup 1830 which can invert to then form the skirt shaped suction cup 1830, as shown in FIG. 30;

FIGS. 32 & 33 illustrate an exemplary embodiment of an engagement catheter 1810 having a cone-shaped suction cup 1830 which can also be retracted to then invert and form the cone-shaped suction cup 1830;

FIGS. 34 & 35 illustrate an exemplary embodiment of an engagement catheter 1810 having a cone-shaped suction cup 1830 which can also be retracted to then invert and form the cone-shaped suction cup 1830;

FIG. 36 illustrates exemplary cone package calculations and exemplary cone material thickness calculations for the cone-shaped suction cup 1830 of the present invention;

FIG. 37 illustrates an end view of the distal end 6100 of the cone-shaped suction cup 1830 of the present invention;

FIG. 38 illustrates a cross-sectional view of the engagement catheter 1810;

FIGS. 39-41 illustrate various exemplary graft embodiments of cone-shaped suction cup 1830 of the engagement catheter 1810;

FIGS. 42 & 43 illustrate perspective views of an engagement catheter 1810 for engaging a tissue 1770 of interest;

FIG. 44 illustrates a side view of a distal end 720 of an engagement catheter 1810 having a flute-shaped suction cup 1830 in a collapsed or retracted configuration;

FIG. 45 illustrates a side view of a distal end 720 of an engagement catheter 1810 having a flute-shaped suction cup 1830 in a deployed or expanded configuration;

FIG. 46 illustrates a cross-sectional view of a distal end 720 of an engagement catheter 1810 adjacent to tissue 1770 of interest;

FIG. 47 illustrates a cross-sectional view of a distal end 720 of an engagement catheter 1810 engaging and applying suction to tissue 1770 of interest;

FIG. 48 illustrates a cross-sectional view of a distal end 720 of an engagement catheter 1810 with internal needle puncturing tissue 1770 of interest;

FIG. 49 illustrates a cross-sectional view of a cross-sectional view of a distal end 720 of an engagement catheter 1810 with internal dilator expanding tissue 1770 of interest;

FIG. 50 illustrates a perspective view of a flute-shaped suction cup 1830 of the present invention with multiple flexible Nitinol tines;

FIGS. 51 & 52 illustrate perspective views of the handle 1900 of an engagement catheter 1810 of the present invention with forward and backward detents 1904 for collapsing or expanding the suction cup 1830;

FIG. 53 illustrates a perspective view of the handle 1900 on the proximal end 710 of an engagement catheter 1810 of the present invention with locking pin 1906;

FIG. 54 illustrates a perspective view of the handle 1900 on the proximal end 710 of an engagement catheter 1810 of the present invention with canted coil spring detent 1912;

FIG. 55 illustrates a perspective view of the handle 1900 on the proximal end 710 of an engagement catheter 1810 of the present invention having a dual hemostasis seal 1914 thereon;

FIG. 56 illustrates a perspective view of a flute or cone-shaped suction cup 1830 of the present invention with longitudinally disposed structural vanes 6010 in a zig-zag configuration

FIG. 57 illustrates a perspective view of a removable handle 1900 on the proximal end 710 of an engagement catheter 1810 having a thumb wheel 1916 for deploying and collapsing the suction cup 1830;

FIG. 58 illustrates a perspective view of a removable handle 1900 on the proximal end 710 of an engagement catheter 1810 which can be removed via handle locking pin 1918;

FIG. 59 illustrates a perspective view of the proximal end 710 of an outer sheath 1800 having a hemostasis valve (or dual valves) on the proximal end 720 thereof;

FIG. 60 illustrates a perspective view of a flute or cone-shaped suction cup 1830 of the present invention with longitudinally disposed structural wire Nitinol vanes 6010 arranged around the circumference of the suction cup 1830 in a looped design similar to that of flower petals;

FIG. 61 illustrates three steps of the process used to form a Nitinol structural vane frame;

FIG. 62 illustrates a perspective view of a Nitinol structural vane frame;

FIG. 63 illustrates a side view of a Nitinol frame formed of curved structural vanes;

FIG. 64 illustrates a side view of a Nitinol frame formed of curved and straight structural vanes;

FIG. 65 illustrates a perspective view of an elastomeric cone-shaped suction cup formed over a Nitinol structural vane frame; and

FIG. 66 illustrates two steps of the process used to form a spring or spiral-shaped Nitinol structural vane frame.

Each of the aforementioned figures pertain to at least one exemplary embodiment of the present disclosure for the subject matter referenced therein.

As such, an overview of the features, functions and/or configurations of the components depicted in the various figures will now be presented. It should be appreciated that not all of the features of the components of the figures are necessarily described and some of these non-discussed features (as well as discussed features) are inherent from the figures themselves. Other non-discussed features may be inherent in component geometry and/or configuration. Furthermore, wherever feasible and convenient, like reference numerals are used in the figures and the description to refer to the same or like parts or steps. The figures are in a simplified form and not to precise scale.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

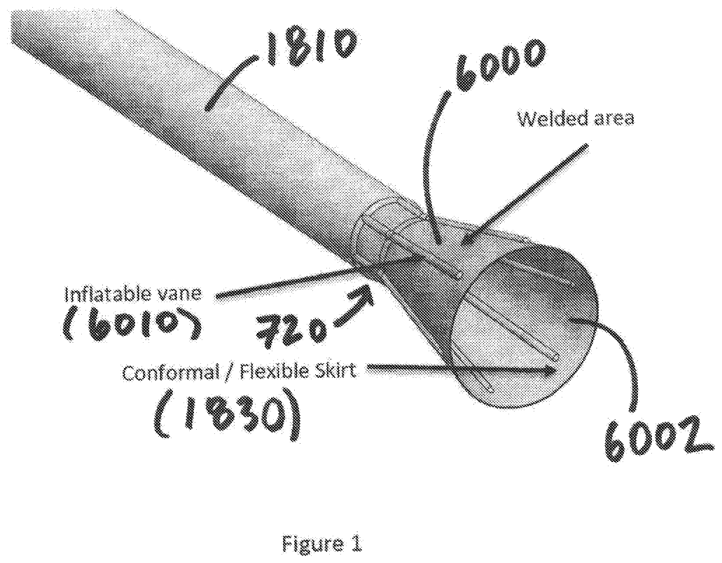

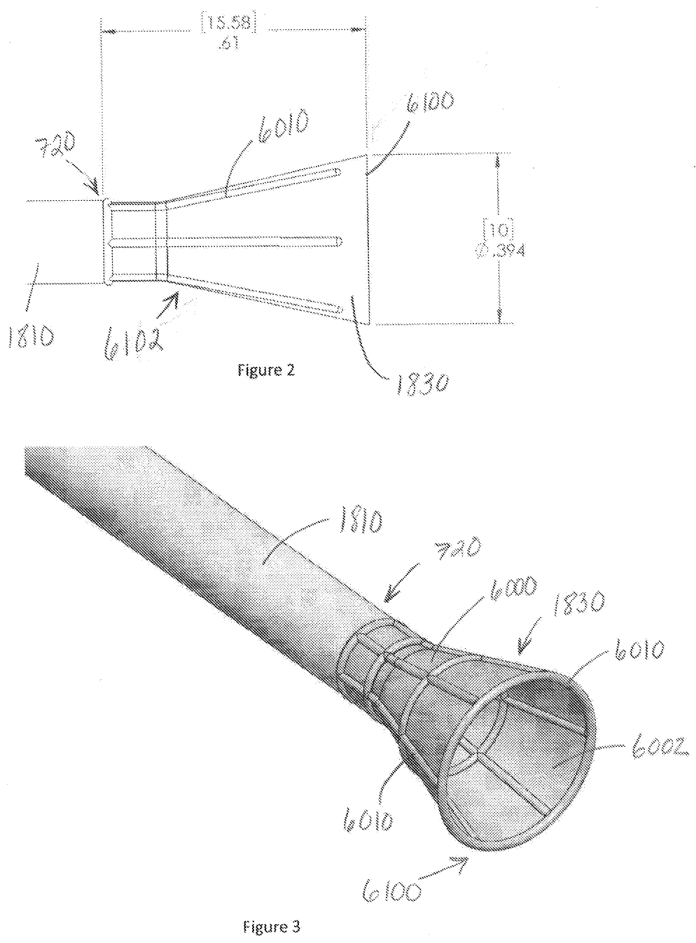

The present disclosure includes various devices, systems, and methods for engaging tissue using suction. As shown in FIG. 1, for example, an exemplary engagement catheter 1810 of the present disclosure comprises a cone-shaped suction cup 1830 at a distal end 720 of engagement catheter 1810 having a configuration allowing cone-shaped suction cup 1830 itself to be inflated and/or deflated as desired. As shown in FIG. 1, cone-shaped suction cup 1830 comprises a first layer of material 6000 and a second layer of material 6002 positioned adjacent to one another, whereby first layer of material 6000 is on a relative outside of cone-shaped suction cup 1830 and whereby second layer of material 6002 is on a relative inside of cone-shaped suction cup 1830. Said layers of material 6000, 6002 may be referred to as film layers, and in various embodiments, such as shown in FIG. 1, layers of material 6000, 6002 are welded or otherwise bonded to one another so to create a plurality of structural vanes 6010, which themselves can be inflated to deploy cone-shaped suction cup 1830 and/or generally provide a structural rigidity, or stiffen, cone-shaped suction cup 1830 so that cone-shaped suction cup 1830 can ultimately engage a tissue 1770 of interest under suction/vacuum. Cone-shaped suction cup 1830 can then be deflated prior to retraction into engagement catheter 1810. In the present embodiment, and potential other embodiments, of the present disclosure, the various structural vanes 6010, and potentially other areas in between first layer of material 6000, and second layer of material 6002, can be inflated and/or deflated as desired. In an exemplary embodiment, best shown in FIG. 2, cone-shaped suction cup 1830 comprises a proximal end 6012 having a circumference substantially similar to an outer circumference of engagement catheter 1810, and a distal end 6100 having a circumference larger than the outer circumference of the engagement catheter 1810.

FIG. 2 shows an exemplary structural geometric rendering of the approximately cone-shaped suction cup 1830 of engagement catheter 1810 of FIG. 1. As shown in FIG. 2, there may be a number of inflatable or structural vanes 6010 in spaced apart relation running longitudinally along the length of cone-shaped suction cup 1830. As shown in FIGS. 1 and 2, the structural vanes 6010 may be disposed along only a portion of the length of cone-shaped suction cup 1830. The structural vanes begin on the distal end 720 of the engagement catheter 1810 or on the proximal end 6102 of the cone-shaped suction cup 1830 and then terminate before reaching the distal end 6100 of cone-shaped suction cup 1830. The proximal end 6102 of the cone-shaped suction cup 1830 is the narrower end adjacent to the distal end 720 of engagement catheter 1810. The distal end 6100 of the cone-shaped suction cup 1830 comprises the larger flared end or rim of the cone-shaped suction cup 1830.

The embodiments shown in FIGS. 3-14 illustrate some of the different exemplary designs for structural vanes 6010, which themselves can be inflated to deploy cone-shaped suction cup 1830 and generally stiffen cone-shaped suction cup 1830 so that cone-shaped suction cup 1830 can ultimately engage a tissue 1770 of interest under suction/vacuum.

FIG. 3 illustrates a cone-shaped suction cup 1830 having a plurality of both radially and longitudinally disposed structural vanes 6010. Structural vanes 6010 may extend along the entire length of cone-shaped suction cup 1830, as well as along a portion of distal end 720 of engagement catheter 1810. There may be any number of structural vanes 6010, having the same or different sizes, shapes, or thicknesses as shown in FIG. 3. As shown in FIG. 3, a structural vane or a plurality of structural vanes 6010 may also be radially disposed around the circumference of the cone-shaped suction cup 1830. Additionally, a structural vane 6010 may also be radially disposed around the perimeter of the distal end 6100 of the cone-shaped suction cup 1830, as shown in FIG. 3. Having the inflatable structural vanes 6010 on the distal end 6100 of cone-shaped suction cup 1830 increases surface area contact with tissue 1770 to improves suction strength and sealing capabilities.

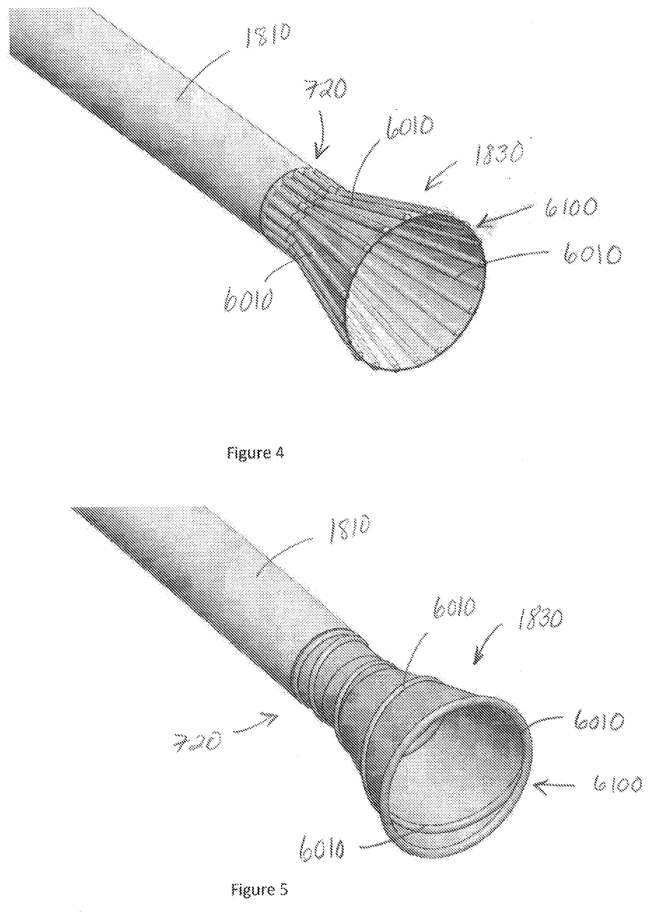

FIG. 4 illustrates a cone-shaped suction cup 1830 having a greater number i.e., density, of longitudinally disposed structural vanes 6010. Structural vanes 6010 extend along the entire length of cone-shaped suction cup 1830 and along a portion of the distal end 720 of engagement catheter 1810. The structural vanes 6010 may terminate at the distal end 6100 of the cone-shaped suction cup 1830. As shown in FIG. 4, the distance between structural vanes 6010 may be decreased to increase the total number of longitudinal structural vanes 6010 on cone-shaped suction cup 1830. The greater number, or higher density, of structural vanes 6010 adds rigidity or stiffness to cone-shaped suction cup 1830 to improve strength of inflation and suction and/or vacuum of a tissue 1770 of interest.

FIG. 5 illustrates the cone-shaped suction cup 1830 having a spiral structural vane or vanes 6010 disposed radially around its circumference. The spiral structural vane 6010 may also be radially disposed around the perimeter of the distal end 6100 of the skirt or suction cup 1830. Having the inflatable structural vanes 6010 on the distal end 6100 of cone-shaped suction cup 1830 increases surface area contact with tissue 1770 to improve suction strength and sealing capabilities.

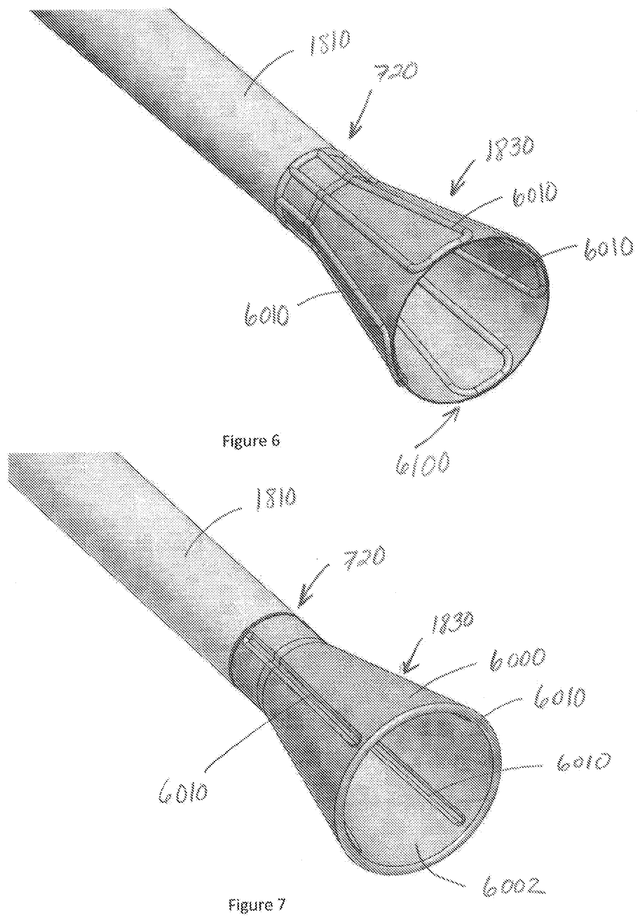

FIG. 6 illustrates a cone-shaped suction cup 1830 having longitudinally disposed structural vanes 6010 which are partially radially attached to one another. Structural vanes 6010 extend along the length of cone-shaped suction cup 1830 and along a portion of the distal end 6100 of engagement catheter 1810. At some areas along the cone-shaped suction cup 1830, the vanes may be disposed radially for a portion of the circumference of the skirt of suction cup 1830 to connect or partially connect the longitudinally disposed structural vanes 6010. Portions of the radially disposed structural vanes 6010 may also be disposed around the perimeter of the distal end 6100 of the cone-shaped suction cup 1830.

FIG. 7 illustrates a cone-shaped suction cup 1830 having large longitudinally disposed structural vanes 6010 welded therein. The larger structural vanes 6010 may further include welds therein tacking layers 6000, 6002 together. The larger size of the structural vanes 6010 may also reduce the overall number of structural vanes 6010 needed to inflate the cone-shaped suction cup 1830. For example, only 2 larger sized structural vanes 6010 may be used. This embodiment may also include a structural vane 6010 located around the perimeter of the distal end 6100 of the cone-shaped suction cup 1830.

FIG. 8 illustrates a flute-shaped suction cup 1830 having a larger inflatable annular area, or balloon 6700, disposed on the distal end 6100 of the flute-shaped suction cup 1830. The larger inflatable balloon area 6700, may be one of the structural vanes 6010 or may be a separate inflatable balloon 6700. The balloon 6700 can be inflated to function similarly to the flute-shaped suction cup 1830 to ultimately engage a tissue 1770 of interest under suction/vacuum. The balloon 6700 can then be deflated prior to retraction into engagement catheter 1810. In addition to the balloon 6700, there may also be a number of structural vanes 6010 disposed longitudinally along the flute-shaped suction cup 1830 and/or along distal end 720 of engagement catheter 1810. The balloon 6700 and the structural vanes 6010 may be separate structures and thus, independently operable. The larger inflatable balloon 6700 increases surface area contact with tissue 1770 to improve suction strength and sealing capabilities with tissue 1770.

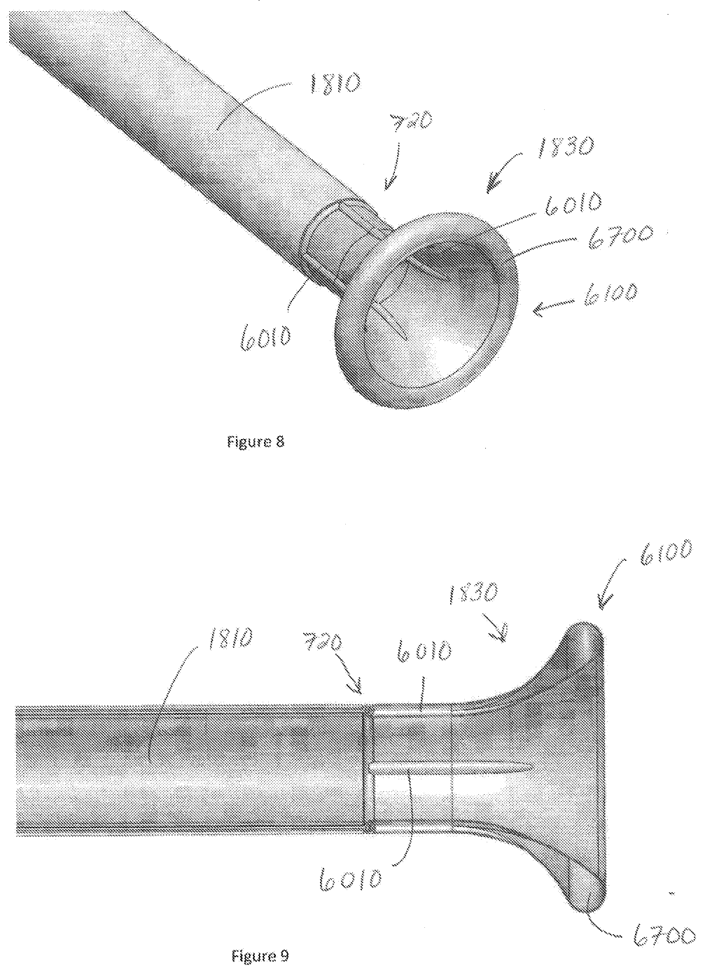

FIG. 9 illustrates a cross sectional view of the embodiment of FIG. 8 having a larger inflatable annular area or balloon 6700 disposed on the distal end 6100 of the flute-shaped suction cup 1830.

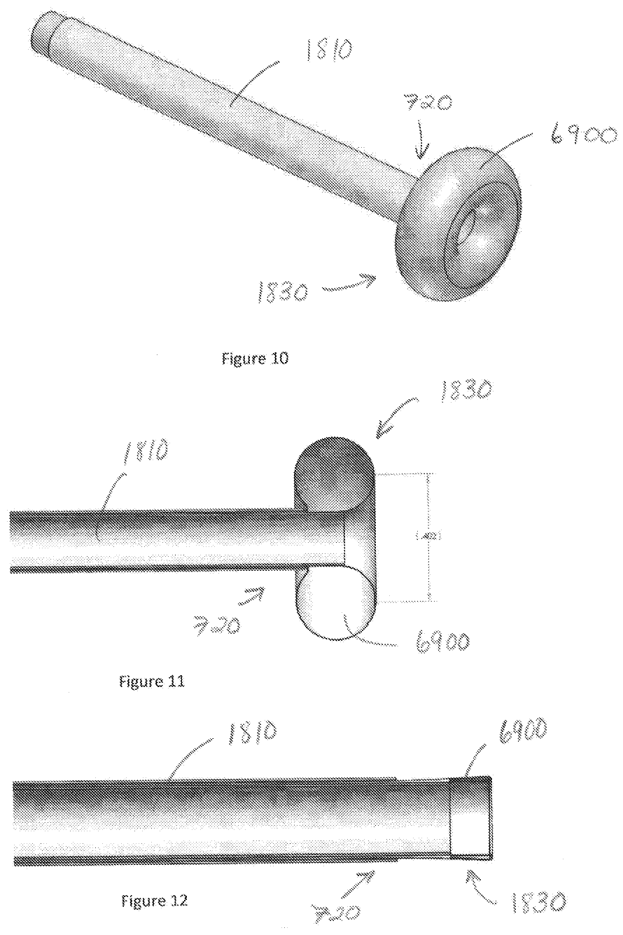

FIG. 10 illustrates an engagement catheter 1810 having an even larger inflatable area or tangent donut 6900 positioned at distal end 720. The tangent donut 6900 may be a large inflatable annular ring. The tangent donut 6900 can be inflated to function similarly to the cone-shaped suction cup 1830 to ultimately engage a tissue 1770 of interest under suction/vacuum. Tangent donut 6900 can then be deflated prior to retraction into engagement catheter 1810. The tangent donut 6900 may be disposed directly on the distal end 720 of engagement catheter.

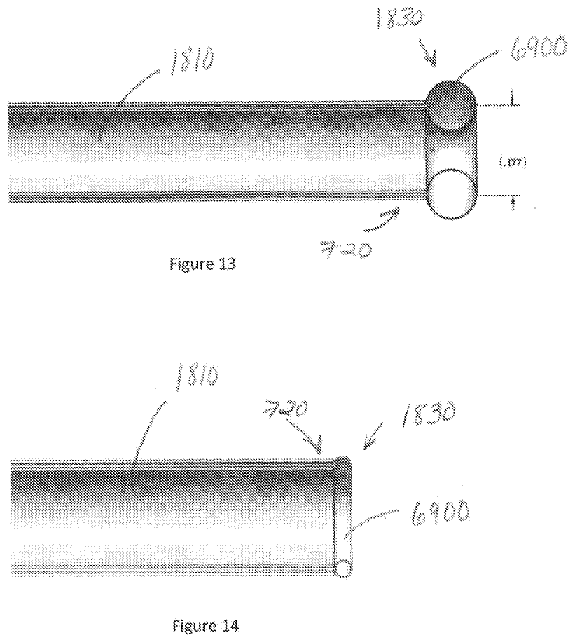

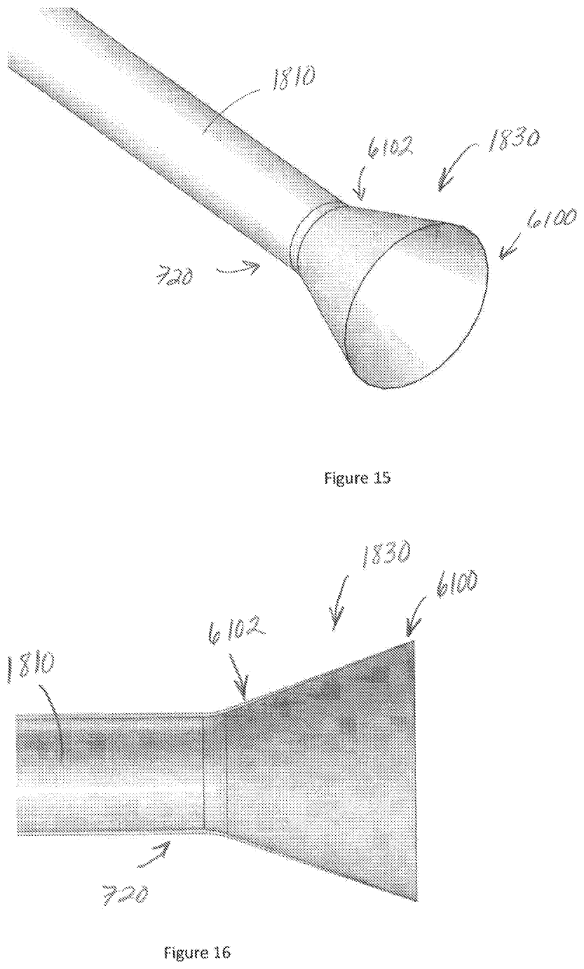

FIG. 11 illustrates a cross-sectional view of the inflated tangent donut 6900 of the embodiment of FIG. 10. FIG. 12 shows a cross-sectional view of the deflated tangent donut 6900 of the embodiment shown in FIG. 10. When tangent donut 6900 is deflated, it may be retracted into the engagement catheter 1810. FIG. 13 illustrates a cross-sectional view of a different embodiment of an inflated tangent donut 6900 disposed on a distal end 720 of the engagement catheter 1810. FIG. 14 illustrates a cross-sectional view of an embodiment of a deflated tangent donut 6900 of the embodiment shown in FIG. 13. FIG. 15 illustrates an embodiment of an engagement catheter 1810 having a dip-molded cone-shaped suction cup 1830 disposed on its distal end 720. The dip-molded cone-shaped suction cup 1830 may be a shape memory material designed to automatically deploy or expand into a cone-shaped suction cup 1830 to ultimately engage a tissue 1770 of interest. When cone-shaped suction cup 1830 is retracted up into sleeve 1800 (not shown in FIG. 15), the cone-shaped suction cup 1830 can be deformed for retraction to fit within engagement catheter 1810. The wall thickness of the dip-molded cone-shaped suction cup 1830 is designed to be uniform or tapered to ensure the cone-shaped suction cup 1830 collapses and fits into the sleeve 1800 of engagement catheter 1810 for retraction. If the wall thickness is tapered, the wall thickness would be greater near the proximal end 6102 of the cone-shaped suction cup 1830 and then become thinner (or taper) toward the distal end 6100 of the cone-shaped suction cup 1830.

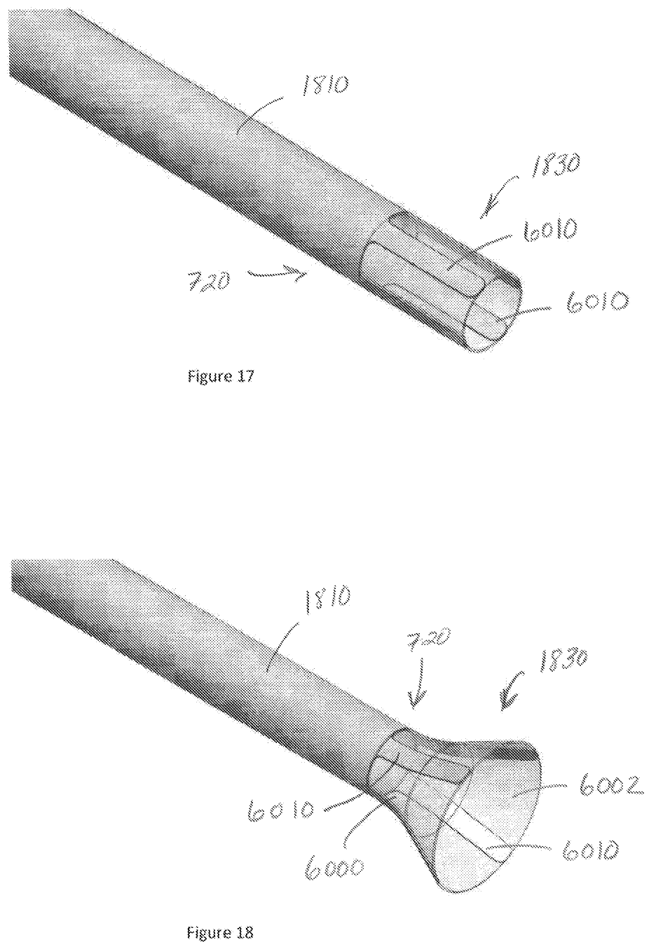

FIG. 16 illustrates a cross-sectional view of the dip-molded cone-shaped suction cup 1830 having a tapered wall thickness. Wall thickness would be greater near the proximal end 6102 of the cone-shaped suction cup 1830 and then become thinner (or taper) toward the distal end 6100 of the cone-shaped suction cup 1830. FIGS. 17 & 18 illustrate an embodiment of an engagement catheter having a Nitinol cone-shaped suction cup 1830 disposed on its distal end 720. The Nitinol cone-shaped suction cup 1830 comprises a first layer of material 6000 and a second layer of material 6002 positioned adjacent to one another, whereby first layer of material 6000 is on a relative outside of cone-shaped suction cup 1830 and whereby second layer of material 6002 is on a relative inside of cone-shaped suction cup 1830. Said layers of material 6000, 6002 may be referred to as film layers, and in various embodiments, are layered via a coating process or welded or otherwise bonded to one another so to create a web around the plurality of structural vanes 6010, which themselves can be expanded to deploy cone-shaped suction cup 1830 and generally provide a structural rigidity, or stiffen, cone-shaped suction cup 1830 so that cone-shaped suction cup 1830 can ultimately engage a tissue 1770 of interest under suction/vacuum.

The structural vanes of this embodiment are formed by cutting a Nitinol tube and heat setting the vanes into a cone shape that will automatically expand cone-shaped suction cup 1830 when deployed from sleeve 1800. The Nitinol cone-shaped suction cup 1830 embodiments are either dip-coated or fused with at least 2 layers 6000, 6002 of an elastomeric film such as polyurethane to fill the interstitial spaces allowing the design embodiments to seal. The Nitinol parts will be electropolished to remove any sharp edges prior to coating or covering. The Nitinol cone-shaped suction cup 1830 is a shape memory material designed to automatically deploy or expand into a cone-shaped suction cup 1830 to ultimately engage a tissue 1770 of interest. When cone-shaped suction cup 1830 is retracted up into sleeve 1800 (not shown in FIGS. 17 & 18), the cone-shaped suction cup 1830 can be deformed for retraction to fit within sleeve 1800.

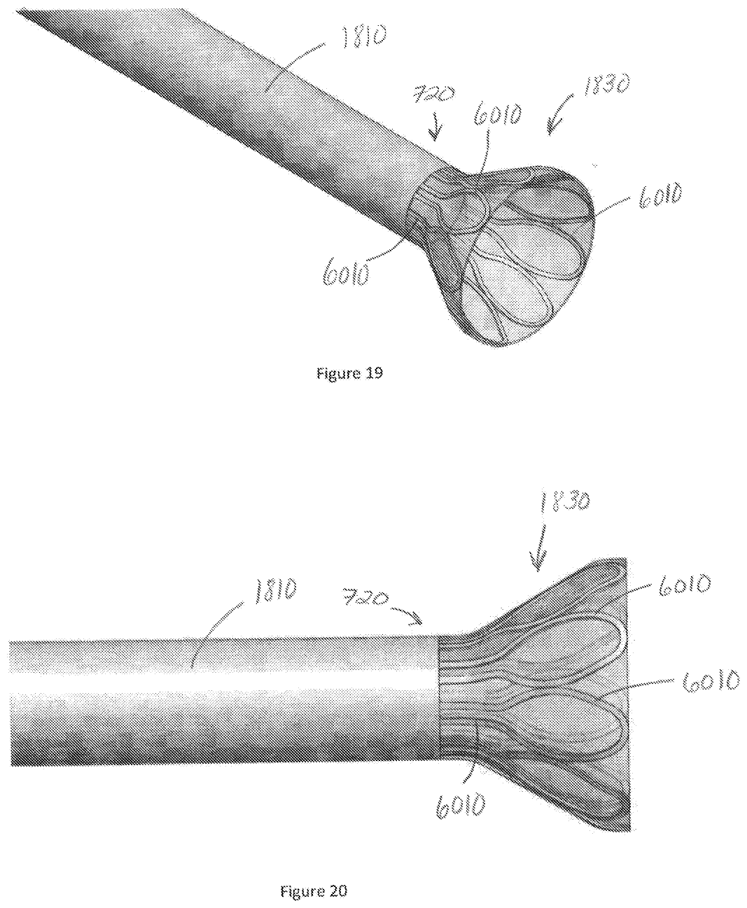

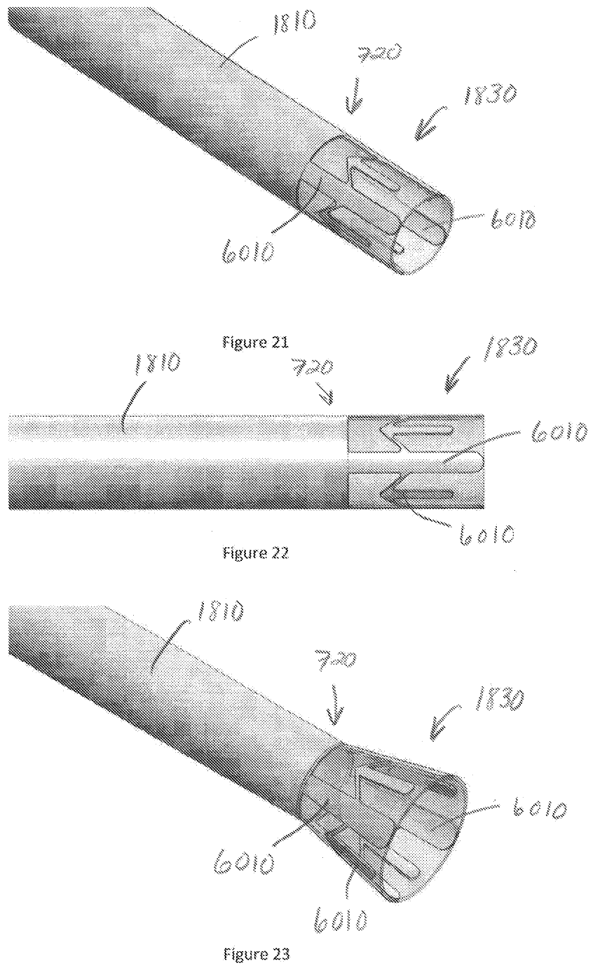

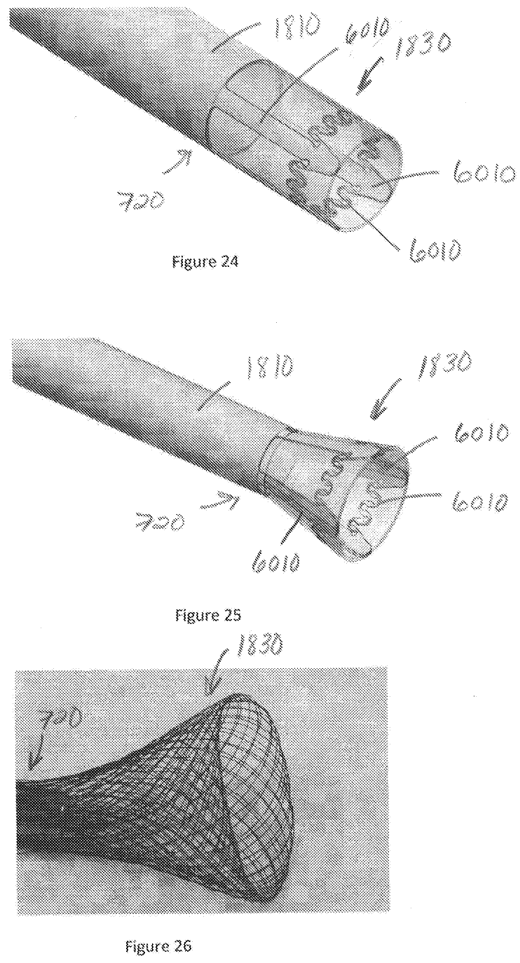

FIG. 17 illustrates an embodiment of an engagement 1810 catheter having a collapsed cone-shaped suction cup 1830. The cone-shaped suction cup 1830 has longitudinally disposed structural vanes 6010 extending from the distal end 720 of the engagement catheter 1810. As shown in FIG. 17, the cone-shaped suction cup 1830 may have 4 or more structural vanes 6010 which extend the entire length of the cone-shaped suction cup 1830. FIG. 18 illustrates an embodiment of the engagement catheter 1810 of FIG. 17 having a cone-shaped suction cup 1830 in an expanded or deployed configuration, so that cone-shaped suction cup 1830 can engage a tissue 1770 of interest. FIGS. 19 & 20 illustrate an exemplary embodiment of an engagement catheter 1810 having a Nitinol suction cup 1830 disposed on its distal end 720 in an expanded configuration. In this embodiment, the suction cup 1830 has longitudinally disposed curving structural vanes 6010 extending along the length of the engagement catheter 1810. The structural vanes 6010 have a uniform thickness and are evenly disposed in a repeating pattern around the circumference of the suction cup 1830. FIGS. 21, 22, & 23 illustrate an exemplary embodiment of an engagement catheter 1810 having a Nitinol cone-shaped suction cup 1830 disposed on its distal end 720. In this embodiment, the cone-shaped suction cup 1830 has a plurality of longitudinally disposed structural vanes 6010 which are connected together by thinner angled linkages 6010. The structural vanes 6010 may have varying thicknesses. FIGS. 21 & 22 shows the cone-shaped suction cup 1830 in a collapsed configuration. FIG. 23 shows the cone-shaped suction cup 1830 in an expanded or deployed configuration. FIGS. 24 & 25 illustrate an exemplary embodiment of an engagement catheter 1810 having a Nitinol cone-shaped suction cup 1830 disposed on its distal end 720. In this embodiment, the cone-shaped suction cup 1830 has a plurality of longitudinally disposed thick structural vanes 6010 which are connected together radially by thinner zig-zagging spring-like linkages 6010. The spring-like linkages 6010 may have varying thicknesses. FIG. 24 shows the cone-shaped suction cup 1830 in a collapsed configuration. FIG. 25 shows the cone-shaped suction cup 1830 in an expanded or deployed configuration.

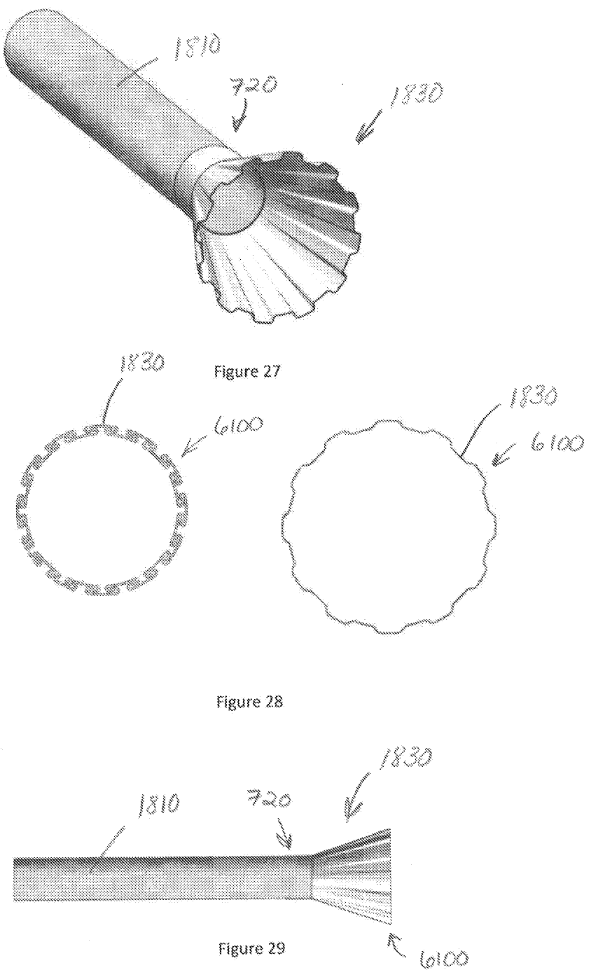

FIG. 26 shows a flute-shaped Nitinol braid 1830 that could be attached to the distal end 720 of an engagement catheter 1810. The Nitinol braid 1830 can be heat set and dip molded or laminated to create a flexible cone or flute-shaped suction cup 1830. FIGS. 27, 28, & 29 illustrate an exemplary embodiment of an engagement catheter 1810 where the suction cup 1830 comprises a self-expanding pleated skirt-shaped cone 1830. The pleated cone may be made of an elastomeric material and the pleats could be molded. The longitudinally disposed pleats facilitate folding or collapse of the skirt-shaped suction cup 1830 for retraction into sleeve 1800. The pleated skirt-shaped suction cup 1830 is designed to automatically deploy or expand into a skirt-shaped suction cup 1830 to ultimately engage a tissue 1770 of interest. When pleated skirt-shaped suction cup 1830 is retracted into sleeve 1800, the skirt or suction cup 1830 can be deformed for retraction to fit within engagement catheter 1810. FIG. 28 shows a cross-sectional view of the distal end 6100 of the skirt or suction cup 1830 having a pleated skirt 1830 in both collapsed (shown in the left FIG. 28) and expanded (shown in the right FIG. 28) configurations.

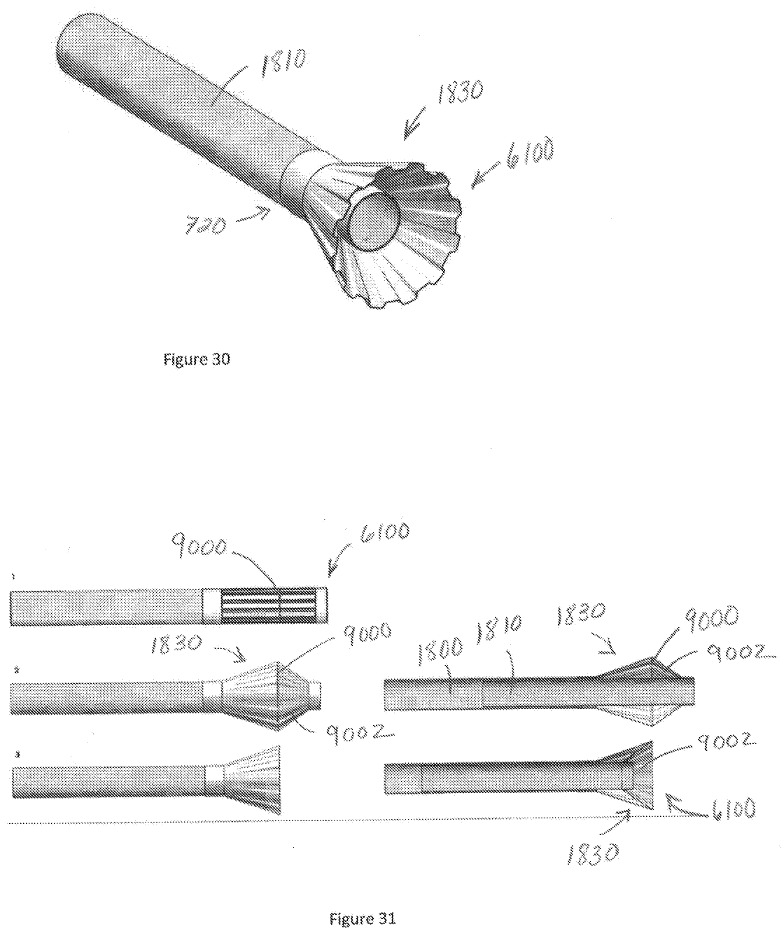

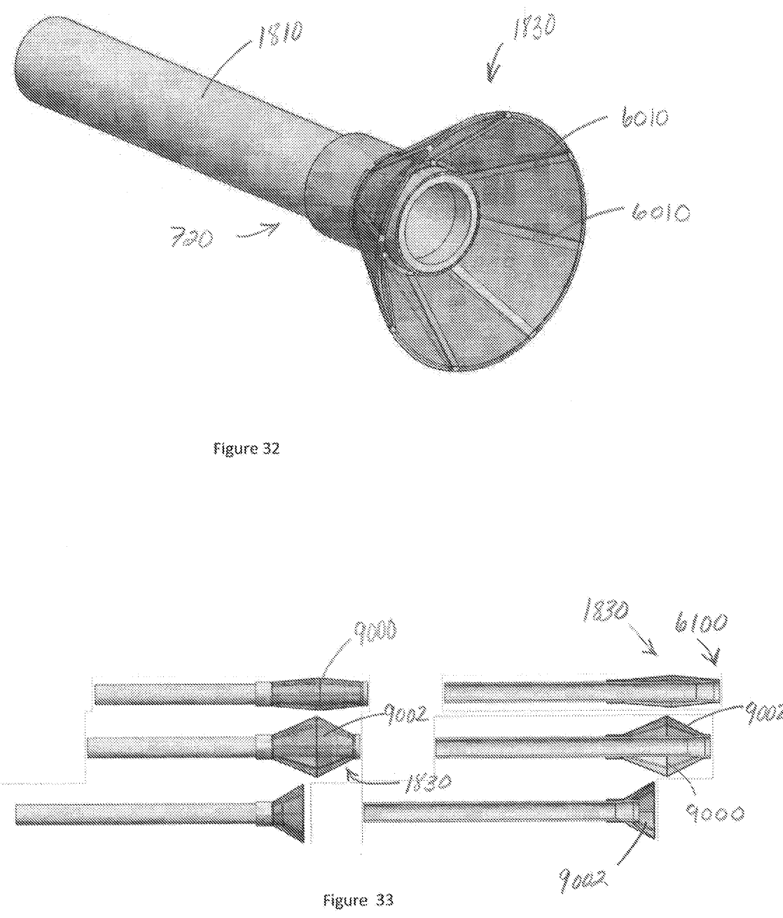

FIGS. 30 & 31 illustrate an exemplary embodiment of an engagement catheter 1810 having a longitudinally pleated sleeve that is attached to an outer sheath at the proximal end 6100 and attached to an inner sheath at the distal end 720. In operation, the outer sheath would be extended while the inner sheath would remain stationary causing the longitudinally pleated sleeve to then invert and form the cone-shaped skirt or suction cup 1830. The inversion could also be achieved when the outer sheath is stationary and the inner sheath is retracted. This embodiment of skirt or the suction cup 1830 has a plurality of longitudinally disposed pleats and a crease 9000 for inversion. FIGS. 32 & 33 illustrate an exemplary embodiment of an engagement catheter 1810 having a sleeve with a plurality of longitudinally disposed Nitinol linkages, attached to an outer sheath at the proximal end and attached to an inner sheath at the distal end. In operation, the outer sheath would be extended while the inner sheath would remain stationary causing the longitudinally disposed Nitinol linkages to then invert and form the cone-shaped skirt or suction cup 1830. The inversion could also be achieved when the outer sheath is stationary and the inner sheath is retracted. This embodiment of the cone-shaped skirt or suction cup 1830 is flexible and non-pleated and has a plurality of longitudinally disposed Nitinol structural linkages 6010 and a crease 9000 for inversion.

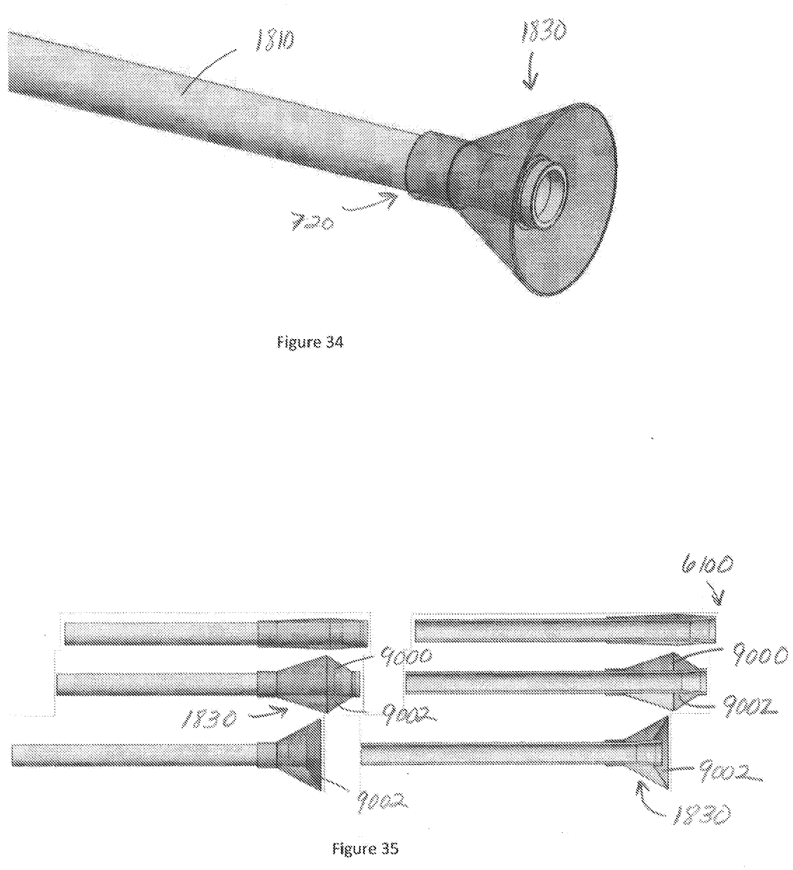

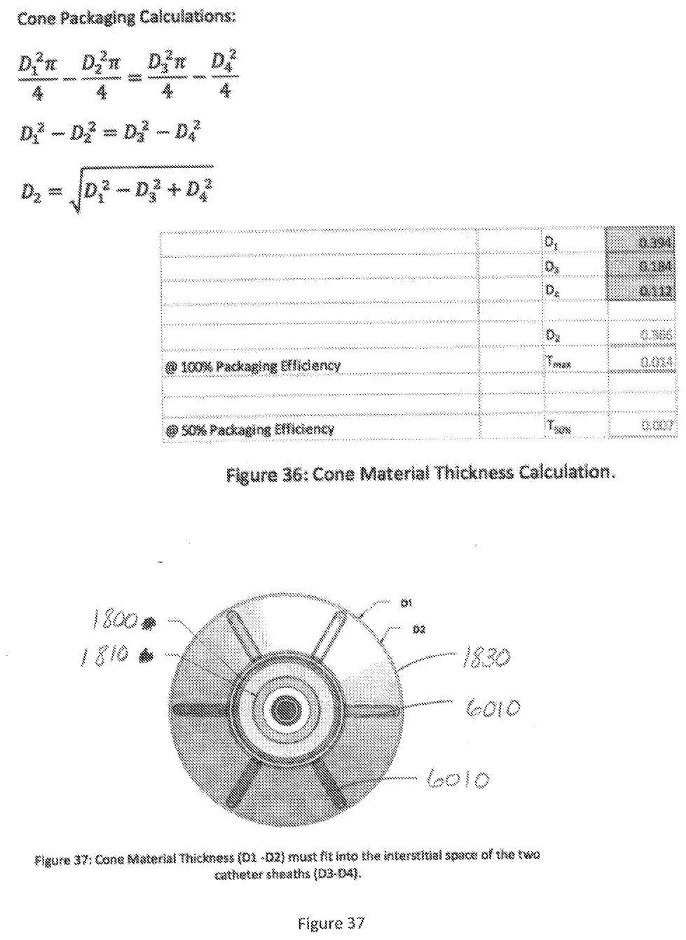

FIGS. 34 & 35 illustrate an exemplary embodiment of an engagement catheter 1810 having a cylindrical sleeve that is attached to an outer sheath at the proximal end and attached to an inner sheath at the distal end. In operation, the outer sheath would be extended while the inner sheath would remain stationary causing the cylindrical sleeve to then invert and form the cone-shaped skirt or suction cup 1830. The inversion could also be achieved when the outer sheath is stationary and the inner sheath is retracted. This embodiment of the cone-shaped suction cup 1830 is flexible and non-pleated and has no structural vanes 6010, but does have a crease 9000 for inversion. FIG. 36 illustrates exemplary cone package calculations and exemplary cone material thickness calculations for the cone-shaped suction cup 1830 of the present invention. FIG. 37 illustrates an end view of the distal end 6100 of the cone-shaped suction cup 1830 of the present invention. This embodiment illustrates the structural vanes 6010 and lumen of the engagement catheter 1810. The cone-shaped suction cup 1830 material thickness must fit into the annular space between both the sleeve 1800 and the engagement catheter 1810.

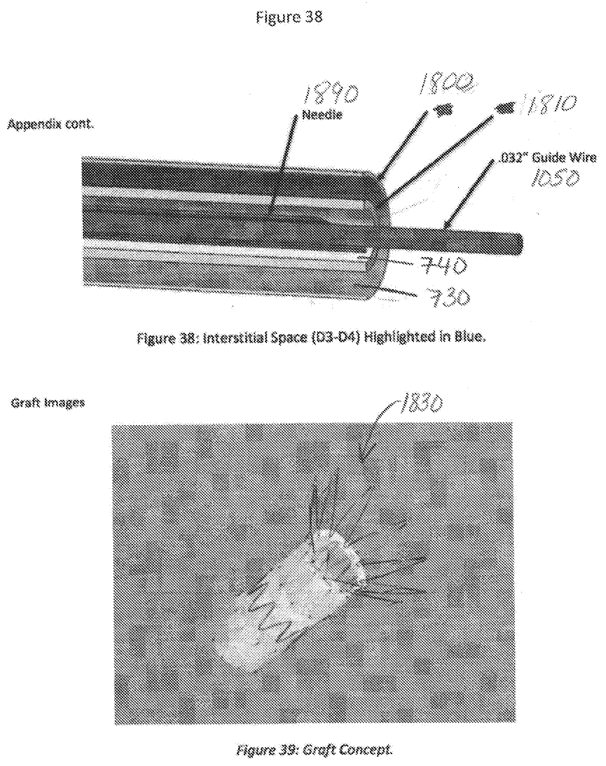

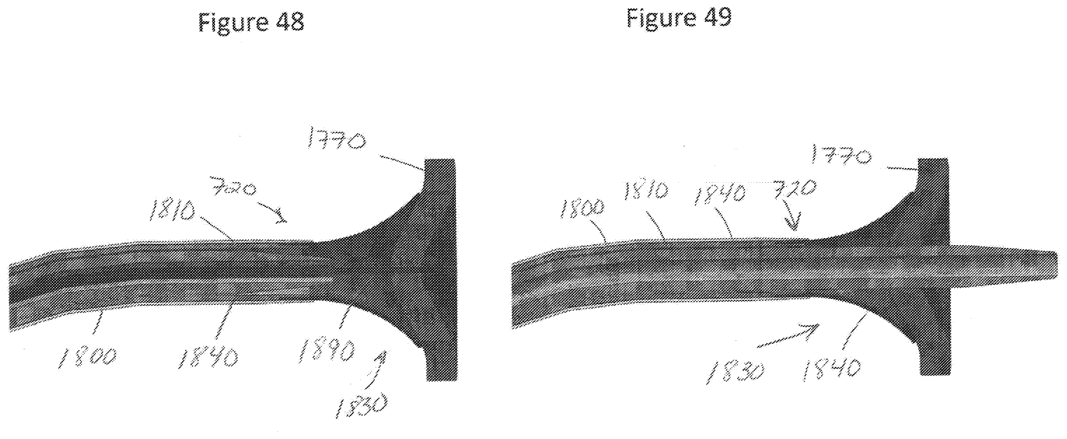

FIG. 38 illustrates a cross-sectional view of the engagement catheter 1810. As shown in FIG. 38, a sleeve 1800 is present around at least a portion of the engagement catheter 1810. Sleeve 1800 may comprise a rigid or flexible tube having a lumen 730 therethrough, appearing around the outside of the engagement catheter 1810 and slideably engaging engagement catheter 1810 such that movement of the sleeve 1800 relative to engagement catheter 1810 can cause cone-shaped suction cup 1830 to be collapsed within or expanded external to sleeve 1800. Engagement catheter 1810 has a lumen 740 therethrough for slideably engaging a dilation catheter 1840 (shown in FIGS. 48 & 49), which may be used to guide a needle 1890 to puncture the tissue 1770 of interest and then dilate the tissue 1770 of interest. After the tissue 1770 of interest has been dilated, the suction cup 1830 is collapsed and the engagement catheter 1810 and sleeve 1800 can be passed through the tissue 1770 of interest to facilitate the delivery of additional catheters or other products (gas, liquid, and or medications, etc). A dilation catheter 1840 is present at least partially within the lumen 740 of engagement catheter 1810, and engagement catheter is placed at least partially within the lumen 730 of sleeve 1800. The dilation catheter 1840 is configured to fit within engagement catheter 1810 and configured for sliding movement relative to engagement catheter 1810. Also shown in FIG. 38, the engagement catheter 1810 may further comprise a needle 1890 having a sharp tip capable of puncturing a tissue 1770. In various embodiments, as shown in FIG. 38, needle lumen is sized and shaped to receive a guide wire 1050 therethrough. The guide wire 1050 can be advanced through a needle 1890 into the atrial or pericardial space to secure the point of entry and guide further insertion of dilation catheter 1840 or another catheter.

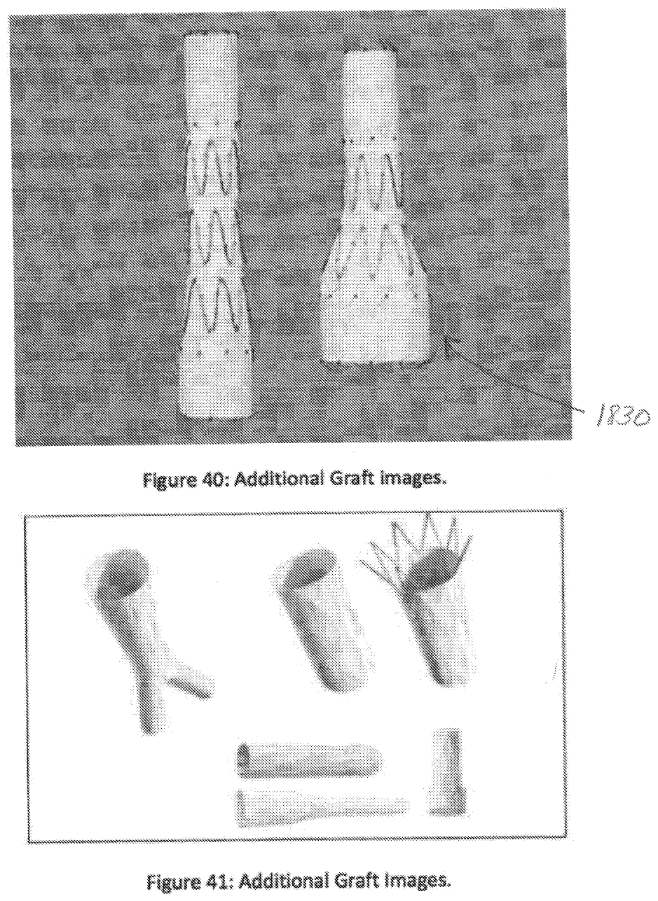

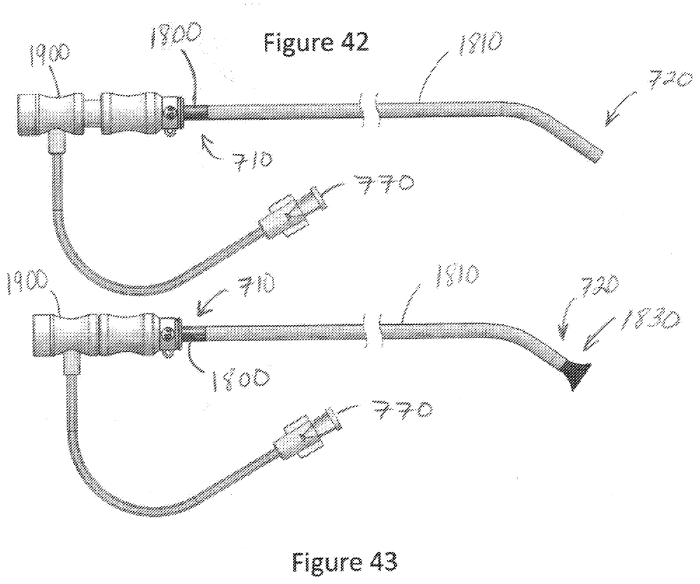

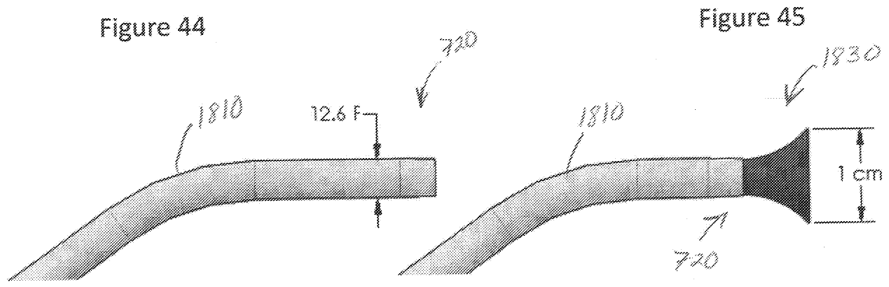

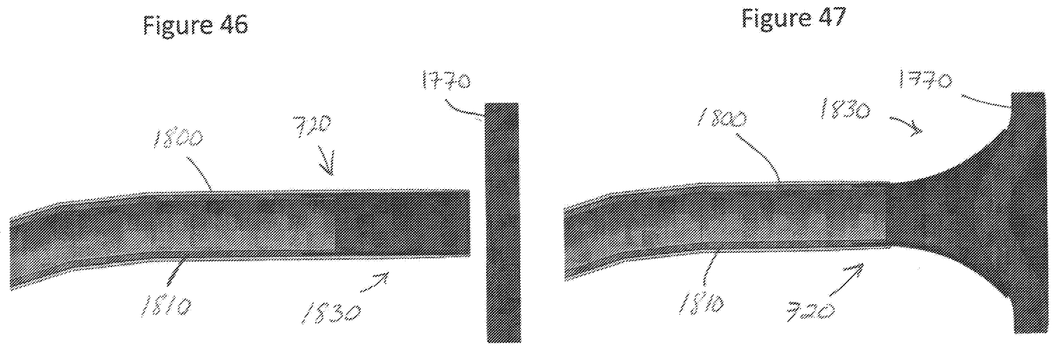

FIGS. 39-41 illustrate various exemplary graft embodiments of cone-shaped suction cup 1830 of the engagement catheter 1810. FIGS. 42 & 43 illustrate perspective views of an engagement catheter 1810 for engaging a tissue 1770 of interest. As shown in FIGS. 42 & 43, an exemplary engagement catheter has a sleeve 1800 slideably engaging engagement catheter 1810, which allows suction cup 1830 to expand when needed. The engagement catheter 1810 may further comprise a vacuum port 770 for connection to an external vacuum source to allow suction cup 1830 to engage a tissue 1770 of interest using suction. The engagement catheter 1810 has a handle 1900 at its proximal end 710 and the flute-shaped suction cup 1830 at its distal end 720. FIG. 42 illustrates the flute-shaped suction cup 1830 collapsed or retracted back into engagement catheter 1810. FIG. 43 illustrates the flute-shaped suction cup 1830 in an expanded or deployed configuration. FIG. 44 illustrates a side view of a distal end 720 of an engagement catheter 1810 having a flute-shaped suction cup 1830 in a collapsed or retracted configuration. FIG. 45 illustrates a side view of a distal end 720 of an engagement catheter 1810 having a flute-shaped suction cup 1830 in a deployed or inflated configuration. FIG. 46 illustrates a cross-sectional view of a distal end 720 of an engagement catheter 1810. In this embodiment, the engagement catheter 1810 is retracted and the flute-shaped suction cup 1830 comprises a Nitinol wire form (and elastomeric coating, or first and second layers 6000, 6002 of material) in a retracted or collapsed configuration. The distal end 720 of the engagement catheter 1810 is adjacent a tissue 1770 of interest. FIG. 47 illustrates a cross-sectional view of a distal end 720 of an engagement catheter 1810. In this embodiment, the engagement catheter 1810 is extended to deploy, expand, and/or inflate flute-shaped suction cup 1830. The flute-shaped suction cup 1830 has engaged tissue 1770 and vacuum or suction is being applied to pull or vacuum tissue 1770 partially into flute-shaped suction cup 1830, as shown in FIG. 47.

FIG. 48 illustrates a cross-sectional view of a distal end 720 of an engagement catheter 1810. In this embodiment, the engagement catheter 1810 is extended to deploy, expand, and/or inflate flute-shaped suction cup 1830 and a dilation catheter 1840 (described further above with regard to FIG. 38) is partially extended to further extend needle 1890 to puncture a tissue 1770 of interest. In one example, a tissue 1770 to be punctured may be the fossa ovalis. As can be seen in FIG. 48, the flute-shaped suction cup 1830 is applying suction or vacuum pressure to the fossa ovalis or tissue 1770, thereby locking the engagement catheter 1810 to the fossa ovalis or tissue 1770 to facilitate puncturing and access through the fossa ovalis or tissue 1770.

FIG. 49 illustrates a cross-sectional view of a distal end 720 of an engagement catheter 1810. In this embodiment, the engagement catheter 1810 is extended to deploy, expand, and/or inflate flute-shaped suction cup 1830 and a dilation catheter 1840 (described further above with regard to FIG. 38) is further extended through the wall of the tissue 1770 of interest, in preparation for delivery of additional catheters or other products such as a liquid, gas, or medication into the atrial or pericardial space. The guide wire 1050 is being advanced through the needle 1890 into the atrial or pericardial space to secure the point of entry and guide further insertion of dilation catheter 1840 or another catheter. In this example, the tissue 1770 to be punctured may be the fossa ovalis. As can be seen in FIG. 49, the flute-shaped suction cup 1830 is applying suction or vacuum pressure to the fossa ovalis or tissue 1770, thereby locking the engagement catheter 1810 to the fossa ovalis or tissue 1770 to facilitate puncturing and access through the fossa ovalis or tissue 1770.

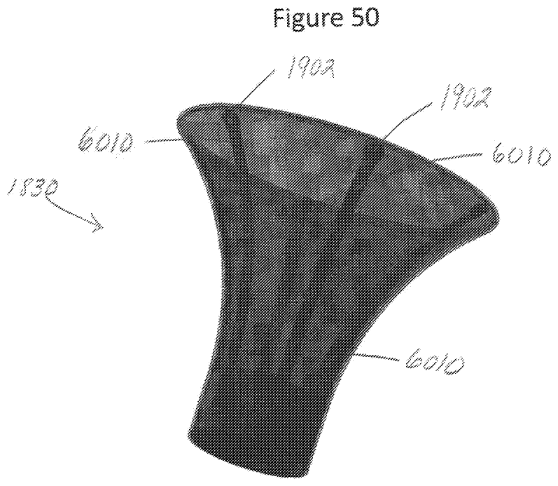

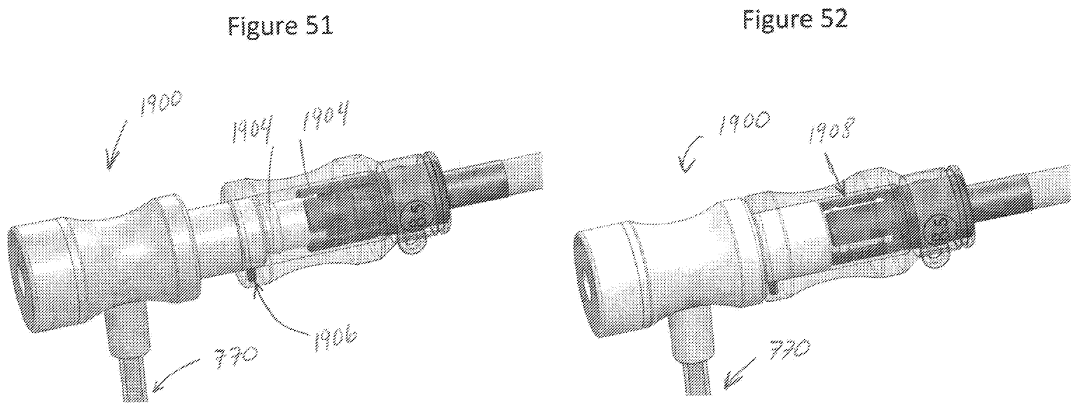

FIG. 50 illustrates a perspective view of a flute-shaped suction cup 1830 of the present invention. In this embodiment, the structural vanes 6010 may comprise longitudinally disposed Nitinol spines having radiopaque markers 1902 positioned thereon. As shown in FIG. 50, the radiopaque markers 1902 may be positioned on the Nitinol structural vanes 6010 at the distal end 6100 of the flute-shaped suction cup 1830. Alternatively, the radiopaque markers 1902 could be positioned at various locations on flute-shaped suction cup 1830 and any number, size or shape of radiopaque markers 1902 may be used. Use of radiopaque markers 1902 is very helpful for determining exact position of flute-shaped suction cup 1830 within a patient using x-ray imaging techniques. FIGS. 51 & 52 illustrate perspective views of the handle 1900 of an engagement catheter 1810 of the present invention. FIG. 51 shows the handle 1900 configured to retract the engagement catheter 1810 using detents 1904 and locking pin 1906. FIG. 52 shows the handle 1900 configured to extend the engagement catheter 1810 using detent collet 1908.

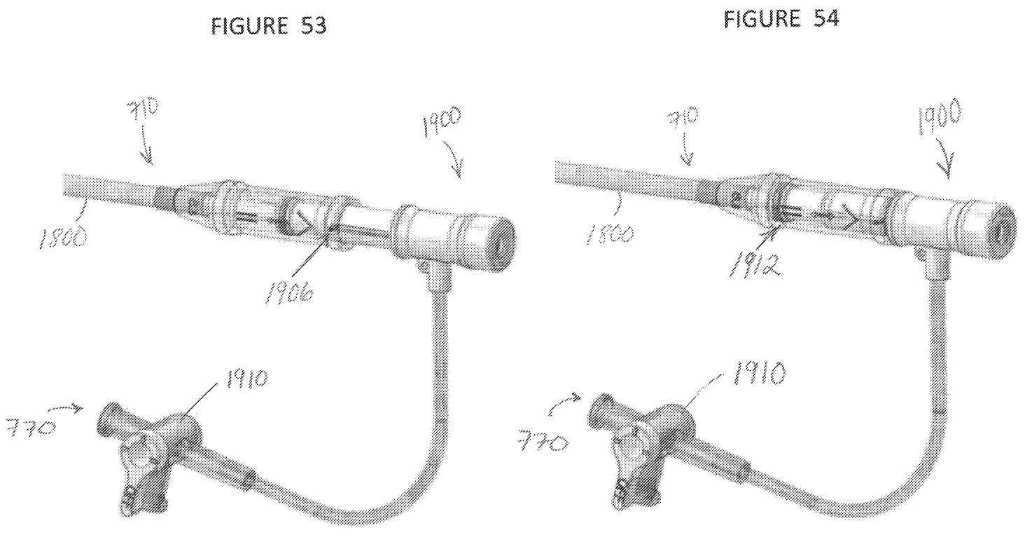

FIG. 53 illustrates a perspective view of the handle 1900 on the proximal end 710 of an engagement catheter 1810 of the present invention. FIG. 53 shows the handle 1900 configured with the outer sheath 1800 extended and the flute-shaped suction cup 1830 in the collapsed configuration using locking pin 1906. FIG. 53 further illustrates use of a three-way stopcock 1910 or valve positioned on vacuum suction port 770 to help the user easily turn suction on and off during a procedure. FIG. 54 illustrates a perspective view of the handle 1900 on the proximal end 710 of an engagement catheter 1810 of the present invention. FIG. 54 shows the handle 1900 configured with the outer sheath 1800 retracted and the flute-shaped suction cup 1830 deployed. This embodiment illustrates a canted coil spring detent 1912. Canted coil spring detents 1912 may comprise forward and backward detents (similar to detents 1904) for collapsing or expanding the suction cup 1830. FIG. 54 also illustrates use of a three-way stopcock or valve positioned on vacuum suction port 770 to help the user easily turn suction on and off during a procedure.

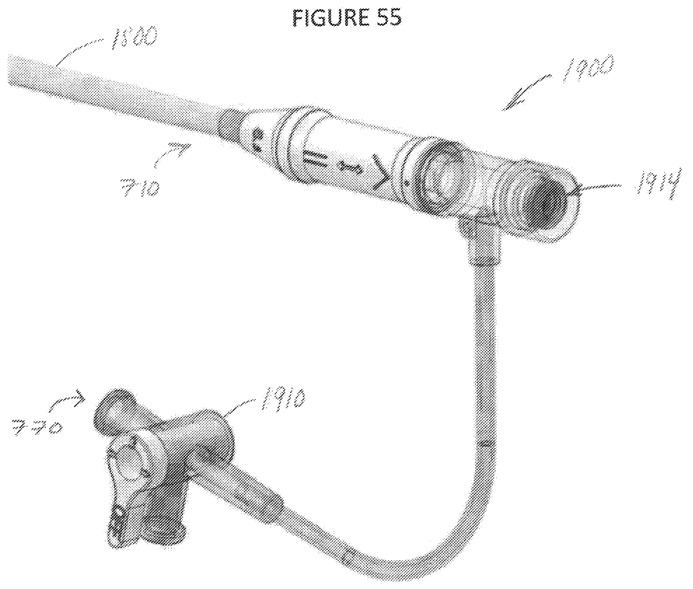

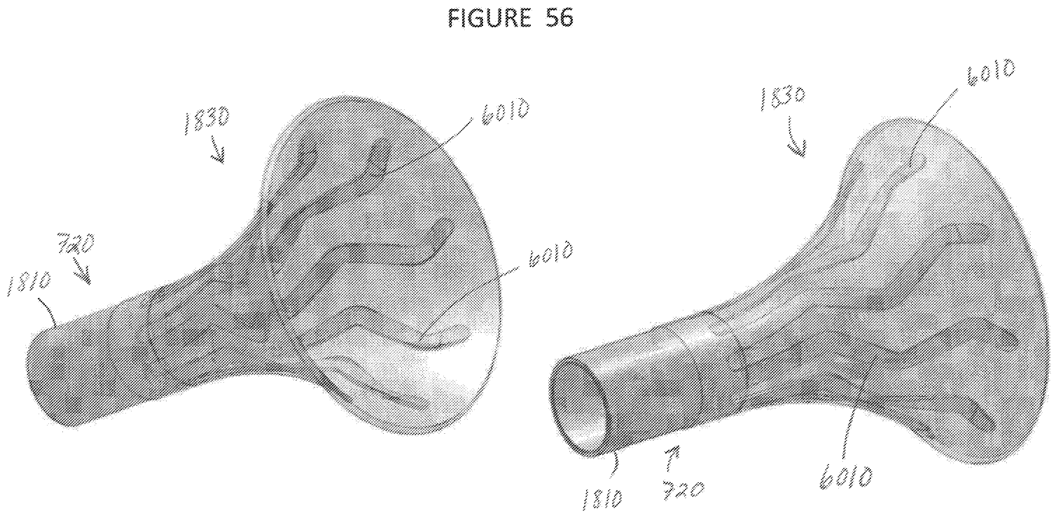

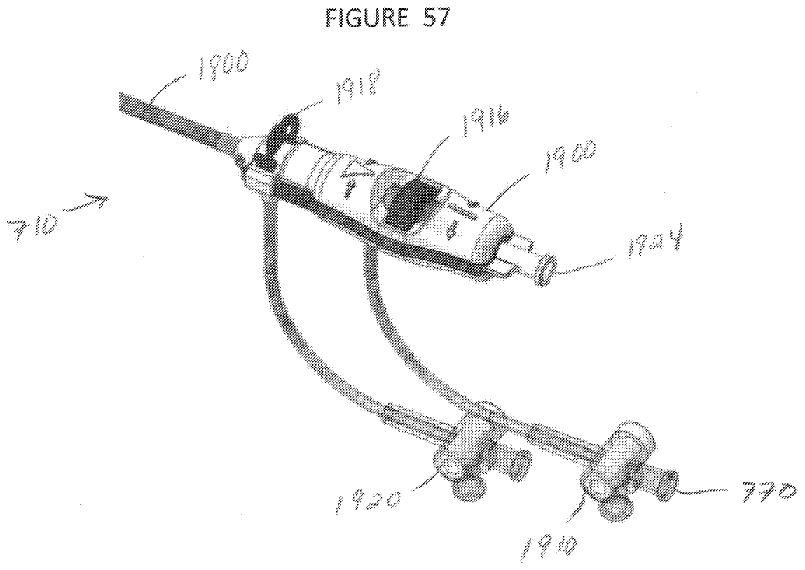

FIG. 55 illustrates a perspective view of the handle 1900 on the proximal end 710 of an engagement catheter 1810 of the present invention. This embodiment of the handle 1900 incorporates dual hemostasis seals 1914. The dual hemostasis seals 1914 are sized to accommodate small guidewires, dilators, catheters, etc. while providing a tight sealing connection. FIG. 56 illustrates an exemplary embodiment of an engagement catheter 1810 having a Nitinol cone-shaped suction cup 1830 disposed on its distal end 720 in a deployed configuration. Cone-shaped suction cups 1830 comprised of Nitinol or other shape memory material are further described herein above with reference to FIGS. 17 and 18. In the embodiment shown in FIG. 56, the cone-shaped suction cup 1830 has a plurality of longitudinally disposed thicker structural vanes 6010 which are disposed in an approximate zig-zagging configuration. The approximately zig-zag shaped vanes 6010 may have varying thicknesses, or a uniform thickness as shown in FIG. 56. The longitudinally disposed zig-zagging vanes 6010 are evenly disposed in a repeating pattern around the circumference of the suction cup 1830. FIG. 57 illustrates a perspective view of the handle 1900 on the proximal end 710 of an engagement catheter 1810 of the present invention having a thumb wheel 1916 for deploying and collapsing the suction cup 1830. A user may spin the thumb wheel 1916 to control deployment of the suction cup 1830 at the distal end 720. In some embodiments, the suction cup 1830 may be slowly deployed and in other embodiments it may be quickly deployed. In this manner, a user may also choose to only partially deploy the suction cup 1830, if desired. A luer port 1924 positioned on proximal end of handle 1900 further provides guidewire and needle access.

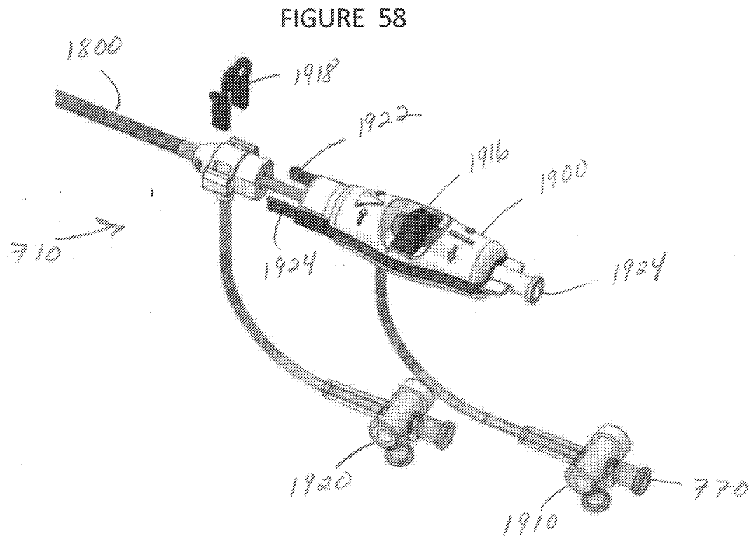

FIG. 57 also illustrates a three-way stopcock 1910 operably coupled to handle 1900 for flushing engagement catheter 1810. The three-way stopcock 1910 also provides vacuum or suction to suction cup 1830 via suction port 770. The outer sheath 1800 may also be operably coupled to a separate three-way stopcock for flushing the outer sheath 1800. FIG. 58 illustrates a detachable handle 1900, which is detachably coupled to the outer sheath 1800 via a removable handle locking pin 1918. The handle locking pin 1918 may have an approximately U-shaped design and operably couples the outer sheath 1800 and handle 1900 together. FIG. 57 illustrates the handle locking pin 1918 in place and FIG. 58 illustrates the handle locking pin 1918 removed. In this embodiment, the handle locking pin 1918, may be removed, such as by squeezing and pulling. Once the handle locking pin 1918 has been removed, the user can squeeze handle connection arms 1922, 1924 together to then pull the handle 1900 away from, and separate from, the outer sheath 1800. Removal of the handle 1900 will also remove the dilator and suction cup 1830 from the patient, providing easier access to the outer sheath 1800 for catheters, guidewires, etc. FIG. 58 also illustrates a three-way stopcock 1910 operably coupled to handle 1900 for flushing engagement catheter 1810. The three-way stopcock 1910 also provides vacuum or suction to suction cup 1830 via suction port 770. The outer sheath 1800 may also be operably coupled to a separate three-way stopcock for flushing the outer sheath 1800.

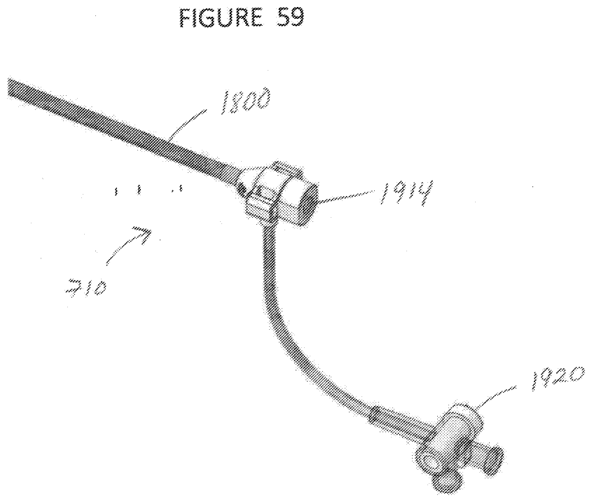

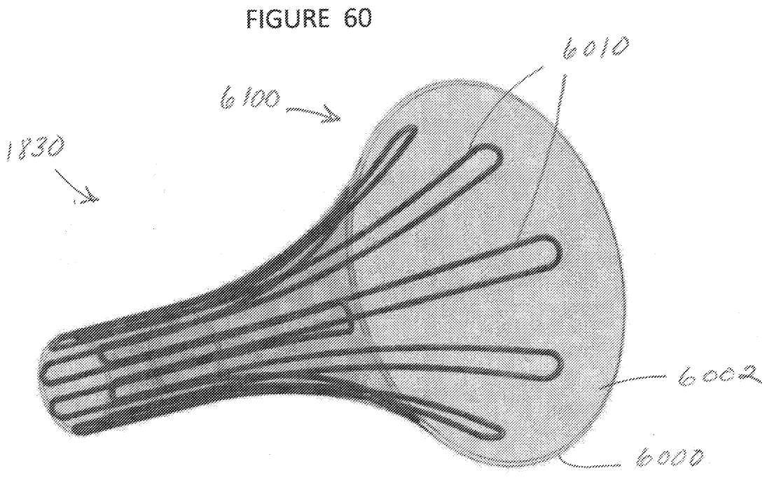

FIG. 59 illustrates a perspective view of the proximal end 710 of an outer sheath design 1800 having a hemostasis valve (or dual valves) 1914 on the proximal end 720 thereof. The hemostasis seal(s) 1914 are sized to accommodate small guidewires, dilators, catheters, etc. while providing a tight sealing connection. FIG. 59 also illustrates the three-way stopcock 1920 operably coupled to outer sheath 1800 for flushing the outer sheath 1800. FIG. 60 illustrates a perspective view of a flute or cone-shaped suction cup 1830 of the present invention with longitudinally disposed structural vanes 6010 arranged around the circumference of the suction cup 1830. In this embodiment, the structural vanes 6010 may comprise wires formed of Nitinol. In this embodiment, the structural vanes 6010 may comprise longitudinally disposed Nitinol wires having loops connecting each longitudinally disposed vane at the distal end 6100 of the flute-shaped suction cup. The loops are connected at the distal end 6100 in a curving manner that looks similar to that of flower petals, as shown in FIG. 60. The wire Nitinol structural vanes 6010 may further be encapsulated in a flexible urethane or other similar deformable and/or collapsible material, such as first and second layers 6000, 6002 described herein above.

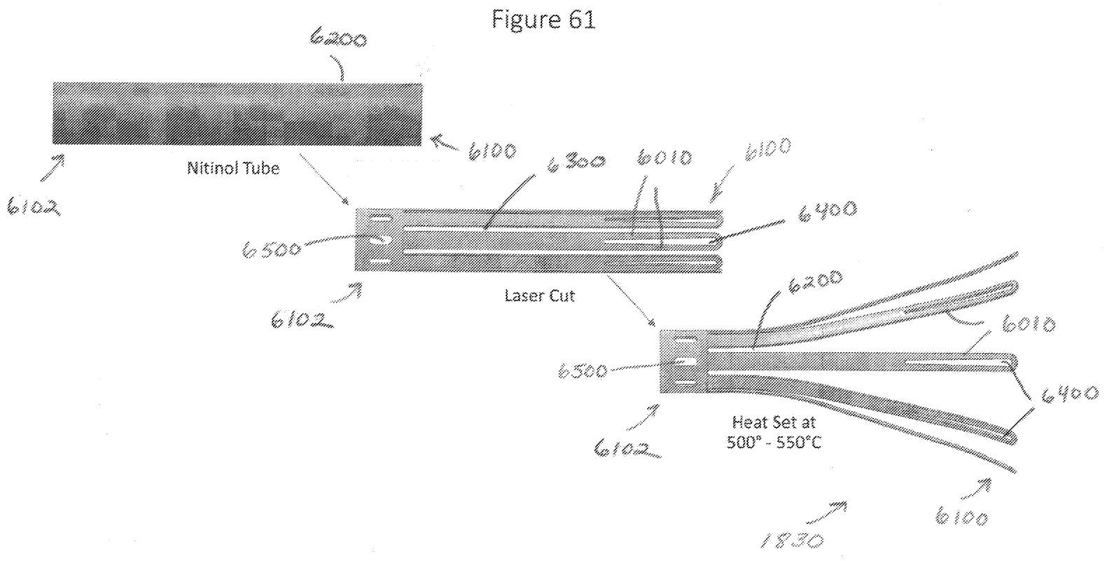

FIG. 61 illustrates the steps of using laser cutting and heat setting processes to form the collapsible Nitinol frame, or structural vane 6010 frame, of a fluted or cone-shaped suction cup 1830. The process or method begins with a Nitinol tube 6200 having a diameter substantially similar to that of engagement catheter 1810, because the proximal end 6102 of the cone-shaped suction cup 1830 will be disposed on, or coupled to, the distal end 720 of the engagement catheter 1810. The proximal end 6102 of the Nitinol tube 6200 may be laser cut to form attachment slots 6500 to be used for coupling attachment to elongated catheter 1810. These attachment slots 6500 may comprise several short elongate slots placed around the circumference of Nitinol tube 6200.

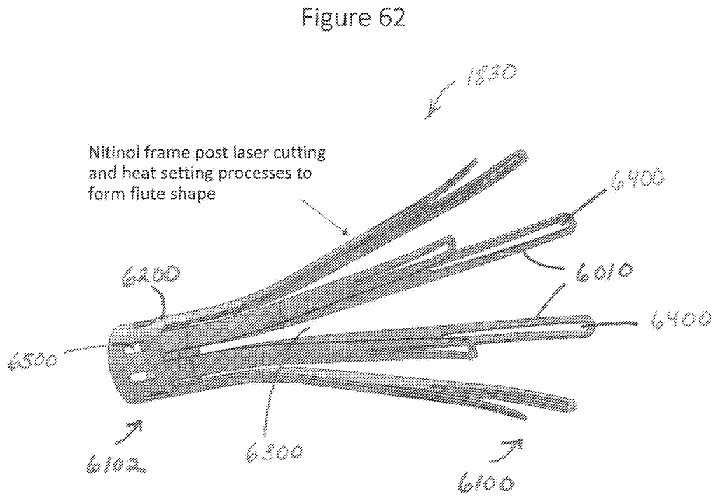

As shown in FIG. 61, the Nitinol tube 6200 is also laser cut along its length at distal end 6100 to form slots 6300, which define several longitudinal structural vanes 6010. The Nitinol structural vanes 6010 themselves may be rounded at the distal ends 6100 and may further comprise additional cuts therein, such as to form elongated teardrop shaped cuts 6400 at their most distal ends 6100. After laser cutting, the section of Nitinol tube 6200 having elongated cuts or slots 6300, 6400, and 6500 therein, may then be heat set at 500° C.-550° C. to form the fluted, flared, or cone-shaped Nitinol structural vanes 6010, best shown in FIG. 62 (and the last image in FIG. 61). These Nitinol structural vanes 6010 form a framework upon which an elastomeric cone-shaped suction cup 1830 will be formed. It should be understood that FIGS. 61 and 62 are exemplary only for the purposes of illustration herein, and the number, size, and shape of cuts and/or slots 6300, 6400, and 6500 and/or structural vanes 6010 may vary from those shown.

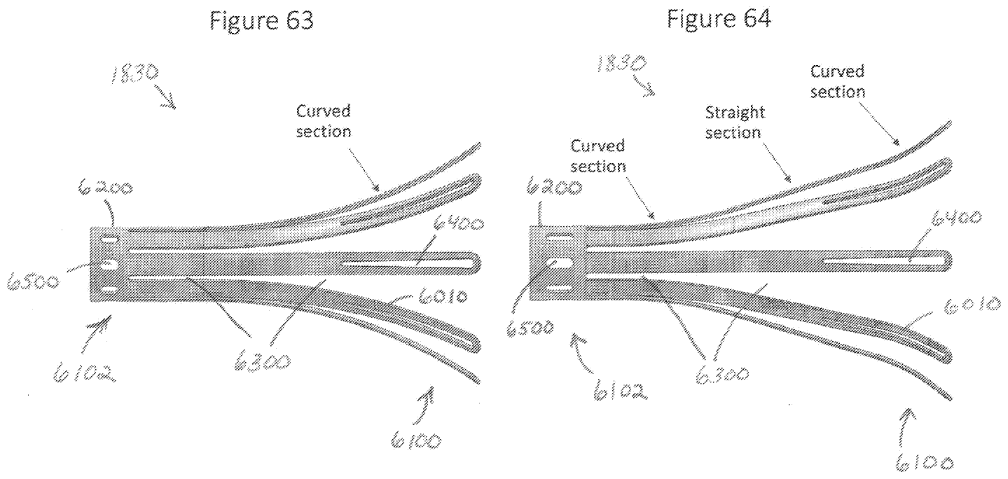

FIGS. 63 and 64 illustrate two embodiments having different Nitinol structural vane 6010 frame designs. The Nitinol structural vanes 6010 may be formed of either curved and/or straight structural vane 6010 shapes. The specific size and shape of the Nitinol structural vanes 6010 may be optimized for flexibility and strength through the disclosed laser cutting and heat setting processes herein. For example, the Nitinol structural vanes 6010 may be wider at the proximal end 6102 of the cone-shaped suction cup 1830 for strength, but may also have wider slots 6400 at the distal end of the in the Nitinol structural vanes 6010 for flexibility. Furthermore, the cross-sectional area of the Nitinol structural vanes 6010 may be reduced or increased along its length as needed to achieve the desired flexibility, strength, etc. As shown in FIG. 63, one embodiment of a Nitinol frame may be comprised entirely of curved Nitinol structural vanes 6010, while another embodiment, shown in FIG. 64, may be comprised of both straight and curved Nitinol structural vanes 6010.

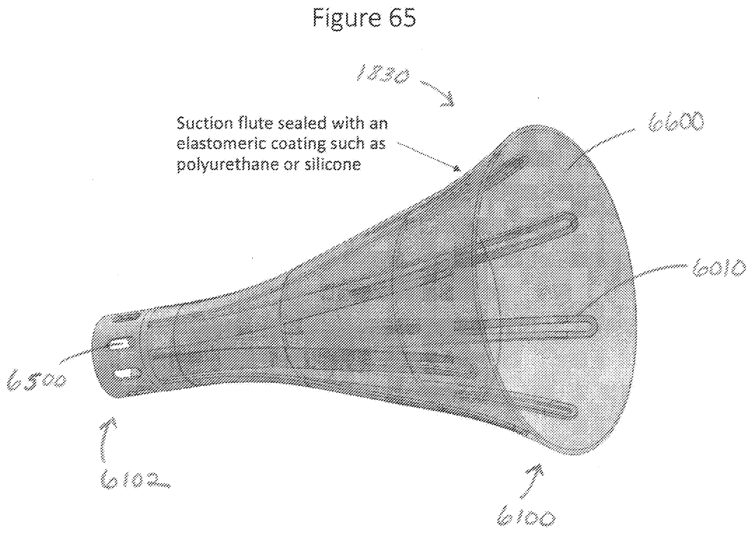

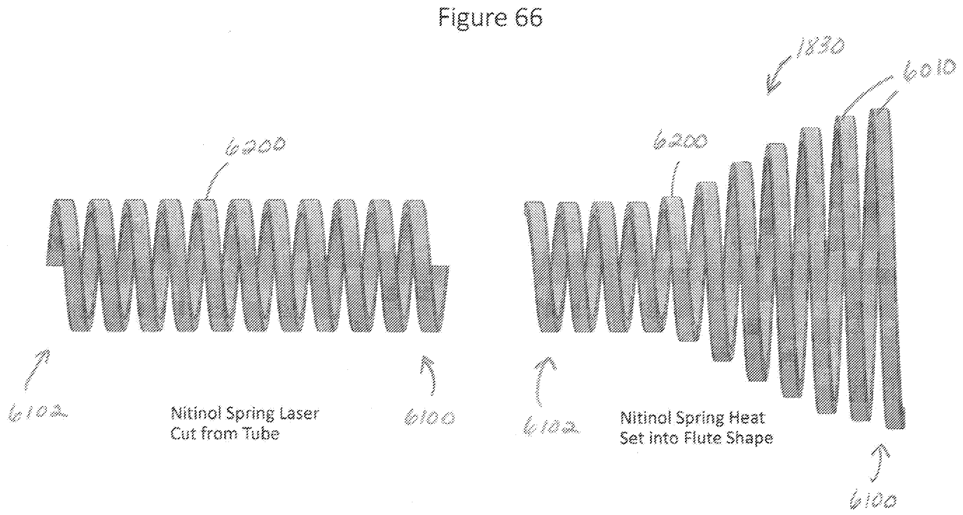

FIG. 65 illustrates an embodiment of a cone-shaped suction cup 1830 formed by adding an elastomeric coating to the Nitinol structural vanes 6010/frame. An elastomeric coating, such as polyurethane or silicone, may be disposed onto the Nitinol structural vanes 6010 to form the cone-shaped suction cup 1830. FIG. 66 illustrates two steps of using laser cutting and heat setting processes to form a spring shaped Nitinol structural vane frame. The process begins with a Nitinol tube 6200 laser cut into a spring or spiral shape and having a diameter substantially similar to that of engagement catheter 1810, because the proximal end 6102 of the cone-shaped suction cup 1830 will be disposed on, or coupled to, the distal end 720 of an engagement catheter 1810. After laser cutting, the Nitinol spring or spiral shaped tube 6200 may then be placed into a fixture or mold (described in more detail below) and heat set at 500° C.-550° C. to form the fluted, flared, or cone-shaped Nitinol structural vane 6010 frame, shown in FIG. 66. The Nitinol structural vane or vanes 6010 may form a spiral or spring-shaped and cone-shaped framework upon which an elastomeric coating or sealer may be applied to form cone-shaped suction cup 1830. With reference now to the methods and processes of forming the cone-shaped suction cups 1830 herein, it should be understood that the elastomeric coating may be applied as two layers of film 6000, 6002 adhered together to form or define structural vanes 6010; or elastomeric coating may be applied as two layers of film 6000, 6002 adhered together over an already formed Nitinol structural vane 6010 frame; or elastomeric coating may be applied as a sealer or coating over an already formed Nitinol structural vane 6010 frame. Further, the figures herein are exemplary only for purposes of illustration and the exact number, sizes, and shapes of the Nitinol structural vanes 6010 and slots 6300, 6400, or 6500 may be changed to adjust the strength, flexibility, and other desired characteristics of the cone-shaped suction cups 1830 desired.

The generally known methods of laser cutting and heat setting or shape setting of Nitinol are followed herein. Specifically, the laser cut Nitinol is put into a fixture or mold which forms and constrains it to the desired final shape. The Nitinol is then heated to the recommended 500° C. temperature for a minimum of 5 minutes in a furnace and then quenched with water to set the shape. The fixture or mold consists of a male side, a female side, and a clamp. The male said is an expansion mandrel that flares the Nitinol to the desired shape. The female side has a cavity on the inside that is an inverted shape of the male side plus the formed Nitinol. The clamp is used to compress the Nitinol between the male and female sides and hold them together with enough force to ensure it won't move during the heating cycle.

An exemplary method of operating the embodiments of engagement catheter 1810 having a flute-shaped suction cup 1830 on its distal end 720 disclosed herein will now be described. An exemplary method of engaging a tissue 1770 of interest to access a space adjacent thereto comprises the step of introducing the engagement catheter 1810 into a mammalian body so that at least part of the system is adjacent to a targeted tissue 1770. The method may further comprise engaging the targeted tissue 1770 using flute-shaped suction cup 1830 of engagement catheter 1810 by applying a vacuum to the engagement catheter 1810 and piercing the targeted tissue 1770 using a needle 1890 to create a tissue aperture. Tissue engagement step may include, but is not limited to, engagement of an atrial wall to ultimately provide access to a pericardial space through an atrial aperture and engagement of an atrial septum to ultimately provide access to a left atrium through an atrial septum aperture, and or various other tissue engagements and/or access that may be possible using various embodiments of the present invention.

While various embodiments of devices and systems and methods for using the same have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A catheter device for engaging tissue using suction, the catheter comprising:
an engagement catheter having a proximal end, a distal end, and a vacuum lumen therethrough operably coupling the proximal and distal ends;
a cone shaped suction cup disposed at the distal end of the engagement catheter to engage a tissue of interest, the cone shaped suction cup comprising:
a first layer of a material and a second layer of a material bonded together, wherein the first layer of material forms an inside wall of the cone shaped suction cup and wherein the second layer of material forms an outside wall of the cone shaped suction cup; and
a plurality of rigid structural vanes, disposed between the first and second layers of material to stiffen the cone shaped suction cup; and
a distal end vacuum port disposed inside of the cone shaped suction cup and operably coupled to the vacuum lumen to suction a tissue of interest.

2. The device of claim 1, wherein the plurality of rigid structural vanes are each inflatable.

3. The device of claim 2, wherein the plurality of inflatable rigid structural vanes are inflated to deploy the suction cup.

4. (canceled)

5. The device of claim 2, wherein the plurality of inflatable rigid structural vanes are disposed longitudinally and radially along a length of the suction cup.

6. The device of claim 2, wherein the plurality of inflatable rigid structural vanes are disposed in a spiral around the circumference of the suction cup.

7. The device of claim 2, further comprising an inflatable rigid structural vane circumferentially disposed around a distal most end of the suction cup.

8. The device of claim 7, further comprising at least two large welded structural vanes disposed longitudinally along a length of the suction cup.

9. (canceled)

10. The device of claim 2, further comprising an inflatable annular area or balloon disposed on a distal most end of the suction cup.

11. The device of claim 10, wherein the inflatable annular area comprises a tangent donut to engage a tissue of interest.

12. The device of claim 2, wherein the plurality of inflatable rigid structural vanes increases surface area contact with tissue to improve suction strength and sealing.

13. The device of claim 1, wherein the cone shaped suction cup is shaped by way of an elongated tube of a shape memory material having a plurality of elongated strips removed from a length of the elongated tube to form elongated shape memory tines at the distal end of the tube, wherein the shape memory tines flare outward away from a central axis to form a fluted distal end;
wherein the first layer of the material and the second layer of the material are effectively one unitary layer of an elastomeric coating; and
wherein the plurality of rigid structural vanes are configured as the elongated shape memory tines.

14.-18. (canceled)

19. The device of claim 1, wherein the cone shaped suction cup is shaped by way of an elongated tube of a shape memory material having a plurality of elongated strips removed from a length of the elongated tube to form elongated shape memory tines at the distal end of the tube, wherein the shape memory tines flare outward away from a central axis to form a fluted distal end.

20.-23. (canceled)

24. The device of claim 1, wherein the suction cup has a tapered wall thickness.

25. The device of claim 1, wherein the suction cup has a wall thickness greater near the proximal end and thinner near the distal end.

26. The device of claim 1, wherein the first and second layers of material are formed of a shape memory material.

27. The device of claim 1, wherein the first and second layers of material are formed of an elastomeric material and the rigid structural vanes are formed of a shape memory material.

28.-40. (canceled)

41. A catheter system for engaging tissue using suction, the catheter system comprising:
an engagement catheter having a proximal end, a distal end, and a vacuum lumen therethrough operably coupling the proximal and distal ends;
a handle operably coupled to the proximal end of the engagement catheter and having a vacuum port operably coupled to the vacuum lumen for providing suction therethrough via an external vacuum source;
a cone shaped suction cup disposed at the distal end of the engagement catheter to engage a tissue of interest, the cone shaped suction cup comprising:
a first layer of a material and a second layer of material bonded together, wherein the first layer of material forms an inside wall of the cone shaped suction cup and wherein the second layer of material forms an outside wall of the cone shaped suction cup; and
a plurality of rigid structural vanes, disposed between the first and second layers of material to stiffen the cone shaped suction cup; and
a distal end vacuum port disposed inside of the cone shaped suction cup and operably coupled to the vacuum lumen to suction a tissue of interest; and
a sleeve slidingly disposed around the engagement catheter and operably coupled to the handle, wherein movement of the sleeve relative to the engagement catheter will collapse or expand the suction cup.

42. The system of claim 41, wherein the suction cup is operable via the handle to retract into the sleeve.

43. (canceled)

44. The system of claim 41, wherein the suction cup further comprising a longitudinally pleated sleeve attached to an outer sheath at the proximal end and attached to an inner sheath at the distal end, wherein extension of the outer sheath, relative to a stationary inner sheath, will invert the longitudinally pleated sleeve to form the suction cup.

45.-47. (canceled)

48. The system of claim 41, wherein the handle further comprises detents and a locking pin operably configured to aid in retraction of the engagement catheter.

49.-66. (canceled)
